SEQUENCE LISTING (27 sequences)

SEQ ID NO: 1 - protein, 616 aa, Aspergillus niger
ATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 2 - protein, 573 aa, Gloeophyllum sepiarium
MYRFLVCALGLAASVLAQSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 3 - protein, 573 aa, Gloeophyllym sepiarium
MYRFLVCALGLAASVLAQSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGSALRSTALITYGNWLLSNGNTSYVTSKLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGIASNAAVATGRYPEDSYQGGNPWYLTTFAVAEQLYDALNVWESQGSLEVTSTSLAFFQQFSSGVTAGTYSSSSSTYSTLTSAIKSFADGFVTINAKYTPSNGGLAEQYSKSDGSPLSAVDLTWSYASALTAFEARNDTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 4 - protein, 573 aa, Gloeophyllum trabeum
MYRFLVCALGLAATVLAQSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSILWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYFDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 5 - protein, 573 aa, Gloeophyllum sepiarium
MYRFLVCALGLAASVLAQSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGIASNAAVATGRYPEDSYQGGNPWYLTTFAVAEQLYDALNVWELQGSLEVTSTSLAFFQQFSSGVTAGTYSSSSSTYSTLTSAIKSFADGFVAINAKYTPSNGGLAEQYSKSDGSPLSAVDLTWSYASALTAFEARNDTQFAGWGAASLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 6 - protein, 573 aa, Gloeophyllum sepiarium
MYRFLVCALGLAATVLAQSVDSYVSSEGPVAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSIYSINSGIASNAAVATGRYPEDSYQGGNPWYLTTFAVAEQLYDALNVWESQGSLEVTSTSLAFFQQFSSGVTAGTYSSSSSTYSTLTSAIKSFADGFVAINAKYTPSNGGLAEQYSKSDGSPLSAVDLTWSYASALTAFEARNDTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 7 - protein, 573 aa, Gloeophyllum sp
MYRFLVCALGLAATVLAQSVDSYVSSEGPVAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSIYSINSGIASNAAVATGRYPEDSYQGGNPWYLTTFAVAEQLYDALNVWESQGSLEVTSTSLAFFQQFSSGVTAGTYSSSSSTYSTLTSAIKSFADGFVAINAKYTPSNGGLAEQYSKSDGSPLSAVDLTWSYASALTAFEARNDTQFAGWGAAGLTVPSSCSGNSGGPTVAVTFNVNAETVWGENIYLTGSVDALENWSADNALLLSSVNYPTWSITVNLPASTAIEYKYIRKNNGAVTWESDPNNSITTPASGSTTENDTWR

SEQ ID NO: 8 - protein, 573 aa, Gloeophyllum trabeum
MYRFLVCALGLAASVLAQSVDSYVSSEGPIAKAGVLANIGPNGSKASGASAGVVVASPSTSDPDYWYTWTRDSSLVFKSLIDQYTTGIDGTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGIASNAAVATGRYPEDSYQGGNPWYLTTFAVAEQLYDALNVWESQGSLEVTSTSLAFFQQFSSGVTAGTYSSSSSTYSTLTSAIKSFADGFVAVNAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 9 - protein, 576 aa, Gloeophyllum trabeum
MYRFLVCALGLLGTVLAQSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGSPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 10 - protein, 576 aa, Gloeophyllum trabeum
MYRFLVCALGLLGTVLAQSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 11 - protein, 618 aa, Talaromyces emersonii
MASLVAGALCILGLTPAAFARAPVAARATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 12 - protein, 559 aa, Gloeophyllum trabeum
QSVDSYVGSEGPIAKAGVLANIGPNGSKASGAAAGVVVASPSKSDPDYWYTWTRDSSLVFKSLIDQYTTGIDSTSSLRSLIDSFVIAEANIQQVPNPSGTLTTGGLGEPKFNVDETAFTGPWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 13 - protein, 928 aa, Bacillus deramificans
DGNTTTIIVHYFRPAGDYQPWSLWMWPKDGGGAEYDFNQPADSFGAVASADIPGNPSQVGIIVRTQDWTKDVSADRYIDLSKGNEVWLVEGNSQIFYNEKDAEDAAKPAVSNAYLDASNQVLVKLSQPLTLGEGASGFTVHDDTANKDIPVTSVKDASLGQDVTAVLAGTFQHIFGGSDWAPDNHSTLLKKVTNNLYQFSGDLPEGNYQYKVALNDSWNNPSYPSDNINLTVPAGGAHVTFSYIPSTHAVYDTINNPNADLQVESGVKTDLVTVTLGEDPDVSHTLSIQTDGYQAKQVIPRNVLNSSQYYYSGDDLGNTYTQKATTFKVWAPTSTQVNVLLYDSATGSVTKIVPMTASGHGVWEATVNQNLENWYYMYEVTGQGSTRTAVDPYATAIAPNGTRGMIVDLAKTDPAGWNSDKHITPKNIEDEVIYEMDVRDFSIDPNSGMKNKGKYLALTEKGTKGPDNVKTGIDSLKQLGITHVQLMPVFASNSVDETDPTQDNWGYDPRNYDVPEGQYATNANGNARIKEFKEMVLSLHREHIGVNMDVVYNHTFATQISDFDKIVPEYYYRTDDAGNYTNGSGTGNEIAAERPMVQKFIIDSLKYWVNEYHIDGFRFDLMALLGKDTMSKAASELHAINPGIALYGEPWTGGTSALPDDQLLTKGAQKGMGVAVFNDNLRNALDGNVFDSSAQGFATGATGLTDAIKNGVEGSINDFTSSPGETINYVTSHDNYTLWDKIALSNPNDSEADRIKMDELAQAVVMTSQGVPFMQGGEEMLRTKGGNDNSYNAGDAVNEFDWSRKAQYPDVFNYYSGLIHLRLDHPAFRMTTANEINSHLQFLNSPENTVAYELTDHVNKDKWGNIIVVYNPNKTVATINLPSGKWAINATSGKVGESTLGQAEGSVQVPGISMMILHQEVSPDHGKK

SEQ ID NO: 14 - protein, 828 aa, Artificial sequence
  Hybrid pullulanase
DSTSTEVIVHYHRFDSNYANWDLWMWPYQPVNGNGAAYEFSGKDDFGVKADVQVPGDDTQVGLIVRTNDWSQKNTSDDLHIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIQVGAAGYEAVNLIPRNVLDSSQYYYSGDDLGNTYTHKATTFKVWAPTSTQVNVLLYNSATGSVTKTVPMTASGHGVWEATVNQNLENWYYMYEVTGQGSTRTAVDPYATAIAPNGTRGMIVDLAKTDPAGWNSDKHITPKNIEDEVIYEMDVRDFSIDPNSGMKNKGKYLALTEKGTKGPDGVKTGIDSLKQLGITHVQLMPVFAFASVDETDPTQDNWGYDPRNYDVPEGQYATNANGTARIKEFKEMVLSLHREHIGVNMDVVYNHTFATQISDFDKIVPEYYYRTDDAGNYTNGSGTGNEIASERPMVQKFIIDSLKYWVNEYHIDGFRFDLMALLGKDTMSKAASELHAINPGIALYGEPWTGGTSALPEDQLLTKGAQKGMGVAVFNDNLRNALDGNVFDSSAQGFATGATGLTDAIKNGVEGSINDFTSSPGETINYVTSHDNYTLWDKIALSNPNDSEADRIKMDELAQAVVMTSQGVPFMQGGEEMLRTKGGNDNSYNAGDTVNEFDWSRKAQYPDVFNYYSGLIHLRLDHPAFRMTTANEINSHLQFLNSPENTVAYELTDHVNKDKWGNIIVVYNPNKTAATINLPSGKWAINATSGKVGESTLGQAEGSVQVPGISMMILHQEVSPDHGKK

SEQ ID NO: 15 - protein, 928 aa, Artificial sequence
  hybrid pullulanase
ASSTEVIVHYHRFDANYANWDLWMWPYQPVNGNGAAYEFSGTDEFGVTADVQVPGDNTQVGLIVRKNDWSQKNTPDDLHIDLSKGHEVWINQGDPTIYYNLNDAEAAAVPAVSNAYLDNEKTVLAKLSSPMTLTDGASGFTVTDETTGEQIPVVSAESANPVTAVLVGDFQQALGASGNWSPDDDHTKLSKINSNLYQFTGTLPAGTYQYKVALDHSWSASYPNNNVNLTVPAGGTKVTFTYIPSTHQVFDSINNPDQTFPSSSAGTQSDLVQLTLASAPDITHDLQVVADGYKGGKILPRNVLNLPRYYYSGNDLGNVYSNKATAFRVWAPTASDVQLLLYNSETGPVTKQLEMQKSDNGTWKLKVPGNLKNWYYLYQVTVNGKTQTAVDPYVRAISVNATRGMIVDLEDTNPPGWKEDHQQTPANPVDEVIYEVHVRDFSIDANSGMKNKGKYLAFTEHGTKGPDGVKTGIDSLKELGINAVQLQPIEEFASIDETQPNMYNWGYDPRNYNVPEGAYATTPEGTARITEFKQLIQSIHKDRIAINMDVVYNHTFSTLISDFDKIVPQYYYRTDDAGNYTNGSGVGNEFATEHPMARKFVLDSLKYWVTQYHIDGFRFDLMALLGKNTMAEASKELHAINPGIVLYGEPWTGGTSGITGDQLLTKGVQKGLGIGVFNDNLRNALDGNVFDSSAQGFATGATGLTDAIKRGVEGSINDFTSSPSETINYVSCHDNYTLWDKIALSNPNDSEADRIKMDELAQAVVMTSQGVPFMQGGEEMLRTKGGNDNSYNAGDTVNEFDWSRKAQYPDVFNYYSGLIHLRLDHPAFRMTTANEINSHLQFLNSPENTVAYELTDHVNKDKWGNIIVVYNPNKTAATINLPSGKWAINATSGKVGESTLGQAEGSVQVPGISMMILHQEVSPDHGKK

SEQ ID NO: 16 - protein, 928 aa, Artificial sequence
  Hybrid pullulanase
ASSTEVIVHYHRFDANYANWDLWMWPYQPVNGNGAAYEFSGTDEFGVTADVQVPGDNTQVGLIVRKNDWSQKNTPDDLHIDLSKGHEVWINQGDPTIYYNLNDAEAAAVPAVSNAYLDNEKTVLAKLSSPMTLTDGASGFTVTDETTGEQIPVVSAESANPVTAVLVGDFQQALGASGNWSPDDDHTKLSKINSNLYQFTGTLPAGTYQYKVALDHSWSASYPNNNVNLTVPAGGTKVTFTYIPSTHQVFDSINNPDQTFPSSSAGTQSDLVQLTLASAPDITHDLQVVADGYKGGKILPRNVLNLPRYYYSGNDLGNVYSNKATAFRVWAPTASDVQLLLYNSETGPVTKQLEMQKSDNGTWKLKVPGNLKNWYYLYQVTVNGKTQTAVDPYVRAISVNATRGMIVDLEDTNPPGWKEDHQQTPANPVDEVIYEVHVRDFSIDANSGMKNKGKYLAFTEHGTKGPDGVKTGIDSLKELGINAVQLQPIEEFASIDETQPNMYNWGYDPRNYNVPEGAYATTPEGTARITEFKQLIQSIHKDRIAINMDVVYNHTFATQISDFDKIVPEYYYRTDDAGNYTNGSGTGNEIAAERPMVQKFIIDSLKYWVNEYHIDGFRFDLMALLGKDTMSKAASELHAINPGIALYGEPWTGGTSALPEDQLLTKGAQKGMGVAVFNDNLRNALDGNVFDSSAQGFATGATGLTDAIKRGVEGSINDFTSSPSETINYVSCHDNYTLWDKIALSNPNDSEADRIKMDELAQAVVMTSQGVPFMQGGEEMLRTKGGNDNSYNAGDTVNEFDWSRKAQYPDVFNYYSGLIHLRLDHPAFRMTTANEINSHLQFLNSPENTVAYELTDHVNKDKWGNIIVVYNPNKTAATINLPSGKWAINATSGKVGESTLGQAEGSVQVPGISMMILHQEVSPDHGKK

SEQ ID NO: 17 - protein, 484 aa, Aspergillus niger
LSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRLYVE

SEQ ID NO: 18 - protein, 583 aa, Rhizomucor pusillus
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFDDASLYHPKCTIDYNNQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 19 - protein, 471 aa, Rhizomucor pusillus
MKFSISLSAAIVLFAAATSLASPLPQQQRYGKRATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSA

SEQ ID NO: 20 - protein, 514 aa, B. stearothermophilus
AAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAW

SEQ ID NO: 21 - protein, 483 aa, Bacillus licheniformis [sequence omitted]

SEQ ID NO: 22 - protein, 483 aa, Bacillus amyloliquefaciens
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 23 - protein, 483 aa, Artificial sequence
  Recombinant alpha-amylase conjugate
AAVNGTLMQYFEWYTPNDGQHWKRLQNDAEHLSDIGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 24 - protein, 483 aa, Artificial sequence
  Bacillus hybrid alpha-amylase
AAVNGTLMQYFEWYTPNDGQHWKRLQNDAEHLSDIGITAVWIPPAYKAISQADVGYGAYDLYDLGEFHQKGTVRTKYGTKGELQSAIKSLHSRDINVYGDVVINHKAGADATEDVTAVEVDPADRNRVISGEHLIKAWTHFHFPGRGSTYSDFKWYWYHFDGTDWDESRKLNRIYKFQGKTWDWEVSNEFGNYDYLMYADFDYDHPDVVAEIKRWGTWYANELQLDGFRLDAVKHIKFSFLRDWVNHVREKTGKEMFTVAEYWSNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 25 - protein, 483 aa, Artificial sequence
  Bacillus hybrid alpha-amylase variant
AAVNGTLMQYFEWYTPNDGQHWKRLQNDAEHLSDIGITAVWIPPAYKAISQADVGYGAYDLYDLGEFWQKGTVRTKYGTKGELQSAIKSLHSRDINVYGDVVINHKAGADATEDVTAVEVDPADRNRVISGEHLIKAWTHFHFPGRGSTYSDFKWYWYHFDGTDWDESRKLNRIYLFQGKTWDWPVSNEFGNYDYLMYADYDYDYPDVVAEITRWGTWYANELQLDGFRLDAVKHIKFSFLRDWVNHVREKTGKEMFTVAEYWSNDLGALENYLNKTNFNHSVFDVPLHYQFHAASTQGGGYDMRKLLNGTVVSKHPLKSVTFVDNHDTQPGQSLESTVQTWFKPLAYAFILTRESGYPSVFYGDMYGTKGDSQREIPALKHKIEPILKARKQYAYGAQHDYFDHHDIVGWTREGVSSVANSGLAALITDGPGGAKWMYVGRQNAGETWHDITGNRSEPVVINSEGWGEFHVNGGSVSIYVQR

SEQ ID NO: 26 - protein, 607 aa, Aspergillus terreus
MKWTSSLLLLLSVIGQATHALTPAEWRSQSIYFLLTDRFGRTDNSTTAACDTSDRVYCGGSWQGIINQLDYIQGMGFTAIWITPVTGQFYENTGDGTSYHGYWQQDIYDLNYNYGTAQDLKNLANALHERGMYLMVDVVANHMGYDGAGNTVDYSVFNPFSSSSYFHPYCLISNYDNQTNVEDCWLGDTTVSLPDLDTTSTAVRNIWYDWVADLVANYSIDGLRVDTVKHVEKDFWPGYNSAAGVYCVGEVYSGDPAYTCPYQNYMDGVLNYPIYYQLLYAFESSSGSISDLYNMISSVASSCKDPTLLGNFIENHDNPRFASYTSDYSQAKNVITFIFLSDGIPIVYAGQEQHYSGGSDPANREATWLSGYSTSATLYTWIATTNQIRSLAISKDAGYVQAKNNPFYSDSNTIAMRKGTTAGAQVITVLSNKGASGSSYTLSLSGTGYSAGATLVETYTCTTVTVDSSGNLPVPMTSGLPRVFVPSSWVNGSALCNTECTAATSISVLFEELVTTTYGENIYLSGSISQLGSWNTASAVALSASQYTSSNPEWYVSVTLPVGTSFQYKFIKKGSDGSVVWESDPNRSYTVPAGCEGATVTVADTWR

SEQ ID NO: 27 - protein, 640 aa, Aspergillus niger
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

(19) United States
(12) Patent Application Publication
(10) Pub. No.: US 2017/0321237 A1
(43) Pub. Date: Nov. 9, 2017

(54) Improved Production of Glucose Syrups

(71) Applicant: Novozymes A/S, Bagsvaerd (DK)

(72) Inventors: Shiro Fukuyama, Chiba (JP); Keiichi Ayabe, Chiba (JP)

(73) Assignee: Novozymes A/S, Bagsvaerd (DK)

(21) Appl. No.: 15/526,886

(22) PCT Filed: Dec. 1, 2015
(86) PCT No.: PCT/EP2015/078223

§ 371 Date: May 15, 2017

(30) Foreign Application Priority Data
Dec. 1, 2014 (EP) 14195685.4

(51) Int. Cl. C12P 19/16; C12P 19/02; C12N 9/44; C13K 1/06; C12N 9/34

(52) U.S. Cl. CPC C12P 19/16; C13K 1/06; C12N 9/2428; C12N 9/2457; C12P 19/02

(57) ABSTRACT

The present invention relates to a method of making glucose syrup from liquefied starch, and to compositions useful therein.

### REFERENCE TO SEQUENCE LISTING

This application contains a Sequence Listing in computer readable form. The computer readable form is incorporated herein by reference.

### FIELD OF THE INVENTION

The present invention relates to methods for production of glucose syrup comprising high % DX from liquefied starch and to compositions comprising enzymes for use therein.

### BACKGROUND OF THE INVENTION

Starch usually consists of about 80% amylopectin and 20% amylose. Amylopectin is a branched polysaccharide in which linear chains alpha-1,4 D-glucose residues are joined by alpha-1,6 glucosidic linkages. Amylopectin is partially degraded by alpha-amylase, which hydrolyzes the 1,4-alpha-glucosidic linkages to produce branched and linear oligosaccharides.

Alpha-amylases are used commercially for a variety of purposes such as in the initial stages of starch processing (e.g., liquefaction). Prolonged degradation of amylopectin by alpha-amylase results in the formation of so-called alpha-limit dextrins that are not susceptible to further hydrolysis by the alpha-amylase. Alpha-amylases (1,4-α-D-glucan glucanohydrolase, EC 3.2.1.1) constitute a group of enzymes which catalyze hydrolysis of starch and other linear and branched 1,4-glucosidic oligo- and polysaccharides.

Branched oligosaccharides can be hydrolyzed into linear oligosaccharides by a debranching enzyme. The remaining branched oligosaccharides can be depolymerized to D-glucose by glucoamylase, which hydrolyzes linear oligosaccharides into D-glucose.

Debranching enzymes which can attack amylopectin are divided into two classes: isoamylases (E.C. 3.2.1.68) and pullulanases (E.C. 3.2.1.41), respectively. Isoamylase hydrolyses alpha-1,6-D-glucosidic branch linkages in amylopectin and beta-limit dextrins and can be distinguished from pullulanases by the inability of isoamylase to attack pullulan, and by their limited action on alpha-limit dextrins.

It is well-known in the art to add isoamylases or pullulanases in starch conversion processes. Pullulanase is a starch debranching enzyme having pullulan 6-glucano-hydrolase activity (EC3.2.1.41) that catalyzes the hydrolysis the α-1,6-glycosidic bonds in pullulan, releasing maltotriose with reducing carbohydrate ends. Usually pullulanase is used in combination with an alpha amylase and/or a glucoamylase.

Glucoamylase (1,4-alpha-D-glucan glucohydrolase, EC 3.2.1.3) is an enzyme, which catalyzes the release of D-glucose from the non-reducing ends of starch or related oligo- and polysaccharide molecules

Commercially, glucoamylases are used to convert starchy material, which is already partially hydrolyzed by an alpha-amylase and e.g. a pullulanase, to glucose in the form of syrup.

Before the enzymatic treatment the starch material, such as whole grains, may be reduced in particle size, e.g., by milling, in order to open up the structure and allowing for further processing. In dry milling whole kernels are milled and used. Wet milling gives a good separation of germ and meal (starch granules and protein) and is often applied at locations where the starch hydrolyzate is used in the production of, e.g., syrups. Both dry and wet milling is well known in the art of starch processing and may be used in a process of the invention.

After milling typically the starch material is liquefied. Liquefaction is carried out in the presence of an alpha-amylase. During liquefaction, the long-chained starch is degraded into branched and linear shorter units (maltodextrins) by the alpha-amylase.

For the production of glucose syrup, the liquefied starch material is saccharified. In a typical saccharification process, maltodextrins produced during liquefaction are converted into dextrose by adding a glucoamylase and a debranching enzyme, such as an isoamylase (U.S. Pat. No. 4,335,208) or a pullulanase. The temperature is lowered to 60° C., prior to the addition of the glucoamylase and debranching enzyme. The saccharification process proceeds for 24-72 hours. Prior to addition of the saccharifying enzymes, the pH is reduced to below 4.5, while maintaining a high temperature (above 95° C.), to inactivate the liquefying alpha-amylase.

For the production of syrup, the enzyme compositions used should at least comprise a glucoamylase and a pullulanase, however, often alpha-amylase activity will also be present, e.g. when using Aspergillus niger glucoamylase the A. niger alpha-amylase from the production host will also be present in the composition. Generally, the presence of alpha-amylase is desirable because it increases the saccharification speed; however it also lowers the dextrose percentage (% DX) in the sachharification product, due to panose formation. Panose is a trisaccharide which is very slowly degraded by glucoamylase, and therefore its formation negatively affects the peak % DX.

From practical and economic perspectives, one would prefer using liquefied starch with a high percentage of dry solid (% DS) as starting material in the saccharification reaction. Unfortunately, there is a trade-off between % DS and % DX: in order to arrive at saccharification products with very high % DX, one will generally need to start from liquefied starch with relatively low % DS. Optimizing the yield in terms of % DX is important for a number of purposes: for example, if the desired end-product is crystalline dextrose, the yield of crystalline dextrose may be improved by up to 3% when the DX is increased by 1%. Even for purposes, where lower % DX is acceptable, it would still be desirable to arrive at that % DX using liquefied starch with higher % DS.

Therefore, there is a need for improved technology for saccharification of liquefied starch.

### SUMMARY OF THE INVENTION

The invention provides compositions and processed for production of improved saccharification products in which it is possible to obtain a higher glucose percentage/conversion rate despite even though the saccharification process is performed using liquefied starch with a high dry solid content.

In particular, the invention provides a composition comprising a glucoamylase and a pullulanase, wherein the ratio of pullulanase activity expressed as NPUN/g to glucoamylase activity expressed as AGU/g is higher than 6.

The invention further provides a process for producing glucose syrup comprising:
i) providing a composition comprising liquefied starch, and adding to said composition a glucoamylase (AMG) and a pullulanase;
ii) subjecting the composition to incubation under conditions allowing starch hydrolysis/saccharification, and subsequently;
iii) adding an alpha-amylase to the said composition and subjecting the composition to incubation under conditions allowing starch hydrolysis/saccharification.

In addition, the invention provides a process for producing glucose syrup from liquefied starch, comprising contacting the liquefied starch with a composition according to the invention.

### BRIEF DESCRIPTION OF DRAWING

FIG. 1 shows the conversion rate (% DX) as function of time in a comparative study where liquefied starch was subjected to saccharification using: (a) a conventional enzyme blend for saccharification comprising glucoamylase, pullulanase and alpha-amylase; (b) a composition according to the present invention comprising glucoamylase (0.25 AGU/gDS), Pullulanase (2.25 NPUN(X)/gDS, and no alpha-amylase; and (c) a composition according to the present invention comprising glucoamylase (0.25 AGU/gDS), Pullulanase (2.25 NPUN(X)/gDS, with addition of alpha-amylase ((0.0225 FAU(F)/gDS) at 24 hours.

### DETAILED DESCRIPTION OF THE INVENTION

### Definitions

Alpha-amylase: Alpha-amylases (1,4-alpha-D-glucan glucanohydrolase, E.C. 3.2.1.1) are a group of enzymes which catalyze the hydrolysis of starch and other linear and branched 1,4 glucosidic oligo- and polysaccharides. Alpha-amylases used according to the present invention may be obtained from fungal or bacterial sources. For purposes of the present invention, fungal alpha amylase activity can be determined as FAU(A) using the alpha amylase assay described in the following paragraph. Activity of bacterial alpha-amylases can be determined as Kilo Novo alpha-amylase Units (KNU) according to the procedure described in the paragraph “Kilo Novo alpha-amylase Units (KNU)” below.

Acid alpha-Amylase Units (FAU(A)): Acid alpha-amylase activity may be measured in FAU(A) (Acid Fungal Alpha-amylase Units). 1 FAU(A) is defined as the amount of enzyme which degrades 5.260 mg starch dry matter per hour under the standard conditions specified in the table “First reaction, starch degradation” below.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

FAU(A), the acid alpha-amylase activity is determined in accordance with the following description. The principle of the reaction is based on the two steps. In the first step the enzyme acid alpha-amylase hydrolyzes starch into different oligosaccharides. In the second step iodine forms a blue complex with starch but not with its degradation products. The intensity of color is therefore directly proportional to the concentration of starch. The activity is determined using reverse colorimetry as a reduction in the concentration of starch under specified analytic conditions.

First reaction, starch degradation

| Substrate | Starch, approx. 0.3 g/L | |
|---|---|---|
| Buffer | Citrate, approx. 0.05M | |
| | CaCl2, 1.85 mM | |
| pH | 2.50 ± 0.05 | |
| Incubation temperature | 37° | C. |
| Reaction time | 180 | seconds |
| Enzyme working range | 0.01-0.04 | FAU(A)/mL |

Second reaction, starch-iodine complex

| | | |
|---|---|---|
| Iodine | 0.0432 | g/L |
| Incubation temperature | 37° | C. |
| Reaction time | 60 | seconds |
| Wavelength | 600 | nm |

Kilo Novo Alpha-Amylase Units (KNU)

The alpha-amylase activity may be determined using potato starch as substrate. The method is based on breakdown of starch in solution by amylase and the fact that starch gives a blue-black colour in presence of iodine. As the enzyme reaction proceeds, aliquots of the reaction are withdrawn and analyzed for their starch content by mixing with an iodine solution. As starch is broken down, the blue-black colour in the presence of iodine fades and gradually turns into a reddish-brown colour. This is compared with a coloured glass standard. The end point is reached when the colour matches the glass standard.

One Kilo Novo alpha amylase Unit (KNU) is defined as the amount of enzyme which, under standard conditions as defined in the “KNU” table below (i.e., at 37° C.+/−0.05; 0.0003 M Ca2+; and pH 5.6) dextrinizes 5260 mg/hour starch dry substance Merck Amylum solubile.

KNU

| | |
|---|---|
| Temperature | 37 ± 0.05° C. |
| pH | 5.6 |
| Substrate concentration | 4.63 mg dry weight/mL |
| Reaction time | 7-20 min, up to 1 hr |
| Ca²⁺ concentration | approx. 0.0003M for reaction |
| | mix containing 2 mL sample |
| | solution |

Glucoamylase (AMG): The term glucoamylase (1,4-alpha-D-glucan glucohydrolase, EC 3.2.1.3) is defined as an enzyme, which catalyzes the release of D-glucose from the non-reducing ends of starch or related oligo- and polysaccharide molecules. For purposes of the present invention, glucoamylase activity is determined as AGU according to the procedure described in the following paragraphs.

Glucoamylase activity (AGU): The Glucoamylase Unit (AGU) is defined as the amount of enzyme, which hydrolyzes 1 micromole maltose per minute in a 0.1 M acetate buffer at an incubation temperature 37° C., a pH of 4.3, a maltose starting concentration of 100 mM, and a reaction time of 6 minutes, thereby generating alpha-D-glucose. The definition applies to an enzyme working range of 0.5-4.0 AGU/mL.

After incubation, the reaction may be stopped with NaOH and the amounts of glucose measured using the following two-step color reaction method: Glucose is phosphorylated by ATP, in a reaction catalyzed by hexokinase. The glucose-6-phosphate formed is oxidized to 6-phosphogluconate by glucose-6-phosphate dehydrogenase. In this same reaction, an equimolar amount of NAD+ is reduced to NADH with a resulting increase in absorbance at 340 nm. Reaction conditions are as specified in the table below:

Color reaction

| Tris | approx. 35 mM | |
|---|---|---|
| ATP | 0.7 | mM |
| NAD⁺ | 0.7 | mM |
| Mg²⁺ | 1.8 | mM |
| Hexokinase | >850 | U/L |
| Glucose-6-P-DH | >850 | U/L |
| pH | approx. 7.8 | |
| Temperature | 37.0° C. ± 1.0° C. | |
| Reaction time | 420 | sec |
| Wavelength | 340 | nm |

Pullulanase: The term “pullulanase” means a starch debranching enzyme having pullulan 6-glucano-hydrolase activity (EC3.2.1.41) that catalyzes the hydrolyses the α-1,6-glycosidic bonds in pullulan, releasing maltotriose with reducing carbohydrate ends. For purposes of the present invention, pullulanase activity is determined as NPUN according to the procedure described in the following paragraph.

Pullulanase activity (NPUN): The NPUN (New Pullulanase Unit Novozymes) is a unit of endopullulanase activity measured in the following procedure.

1 NPUN=One pullulanase unit (NPUN) is defined as the enzyme amount, which releases reducing ends equivalent to 0.35 μmol glucose per minute under the standard conditions specified in the table “First reaction, pullulan degradation” below.

In the first reaction, the substrate is equally present in both sample main and sample blank. However, the reaction of sample main is performed at pH 5.0, while there is no reaction in the sample blank at pH 9.6, where neither pullulanases nor amyloglucosidases (glucoamylase) are enzymatically active.

First reaction, pullulan degradation

| Substrate | BH4 reduced pullulan, 5.3 g/L | |
|---|---|---|
| Buffer (main) | Acetate, approx. 0.1M | |
| | EDTA, 5.3 mM | |
| | Acarbose, 0.018% | |
| | (if sample contains glucoamylase) | |
| pH (main) | 5.0 | |
| Buffer (blank) | CHES, 42 mM | |
| | acetate, 17 mM | |
| | EDTA, 5.3 mM | |
| pH (blank) | 9.6 | |
| Incubation temperature | 50° | C. |
| Reaction time | 540 | seconds |
| Enzyme working range | 0.03-0.15 | NPUN/mL |

In the second reaction, the pH is adjusted to approx. 9.6 and the glucose in samples is phosphorylated to non-reducing D-glucose-6-phosphate by glucokinase, which has optimal activity and stability in this range and is specific to glucose at pH 9 (ref. Goward, Biochem. J. 1986, 237, pp 415-420). This step depends on identical pH in sample main and sample blank to remove equal amounts of glucose in both.

Second reaction, background glucose elimination

| Substrate | glucose in sample, after first reaction | |
|---|---|---|
| Buffer | CHES, 58 mM (main) or 76 mM (blank) | |
| | acetate, 43 mM (main) or 7.2 mM (blank) | |
| | EDTA, 2.2 mM | |
| | ATP, 1.11 mg/ml | |
| | MgCl2, 4.4 mM | |
| Glucokinase | 0.11 | U/ml |
| pH | approx. 9.6 | |
| Incubation temperature | 50° | C. |
| Reaction time | 720 | seconds |

The second reaction is stopped by and alkaline reagent >pH 11 containing PAHBAH (pHydroxy benzoic acid hydrazide) and bismuth, which complexes with reducing sugars to produce color detected at 405 nm. The produced color is proportional to the pullulanase activity.

Third reaction, PAHBAH-Bi reaction

| Substrate | maltotriose formed by pullulanase, | |
|---|---|---|
| | after second reaction | |
| PAHBAH | 56 | mM |
| Tartrate | 75 | mM |
| Bi³⁺ | 6.0 | mM |
| NaOH | 195 | mM |
| pH | alkaline | |
| Incubation temperature | 50° | C. |
| Reaction time | 1000 | seconds |
| Wavelength | 405 | nm |

Mature polypeptide: The term “mature polypeptide” means a polypeptide in its final form following translation and any post-translational modifications, such as N-terminal processing, C-terminal truncation, glycosylation, phosphorylation, etc. According to some embodiments, the mature polypeptide of SEQ ID NO: 2 consists essentially of amino acids 18 to 573 of SEQ ID NO: 2, the mature polypeptide of SEQ ID NO: 3 consists essentially of amino acids 18 to 573 of SEQ ID NO: 3, the mature polypeptide of SEQ ID NO: 4 consists essentially of amino acids 18 to 573 of SEQ ID NO: 4, the mature polypeptide of SEQ ID NO: 5 consists essentially of amino acids 18 to 573 of SEQ ID NO: 5, the mature polypeptide of SEQ ID NO: 6 consists essentially of amino acids 18 to 573 of SEQ ID NO: 6, the mature polypeptide of SEQ ID NO: 7 consists essentially of amino acids 18 to 573 of SEQ ID NO: 7, the mature polypeptide of SEQ ID NO: 8 consists essentially of amino acids 18 to 573 of SEQ ID NO: 8, the mature polypeptide of SEQ ID NO: 9 consists essentially of amino acids 18 to 576 of SEQ ID NO: 9, the mature polypeptide of SEQ ID NO: 10 consists essentially of amino acids 18 to 576 of SEQ ID NO: 10, the mature polypeptide of SEQ ID NO: 11 consists essentially of amino acids 21 to 618 of SEQ ID NO: 11, the mature polypeptide of SEQ ID NO: 12 consists essentially of amino acids 1 to 559 of SEQ ID NO: 12, the mature polypeptide of SEQ ID NO: 13 consists essentially of amino acids 1 to 928 of SEQ ID NO: 13, the mature polypeptide of SEQ ID NO: 14 consists essentially of amino acids 1 to 828 of SEQ ID NO: 14, the mature polypeptide of SEQ ID NO: 15 consists essentially of amino acids 1 to 928 of SEQ ID NO: 15, the mature polypeptide of SEQ ID NO: 16 consists essentially of amino acids 1 to 928 of SEQ ID NO: 16, the mature polypeptide of SEQ ID NO: 17 consists essentially of amino acids 1 to 484 of SEQ ID NO: 17, the mature polypeptide of SEQ ID NO: 18 consists essentially of amino acids 1 to 583 of SEQ ID NO: 18, the mature polypeptide of SEQ ID NO: 19 consists essentially of amino acids 22 to 471 of SEQ ID NO: 19, the mature polypeptide of SEQ ID NO: 20 consists essentially of amino acids 1 to 514 of SEQ ID NO: 20, the mature polypeptide of SEQ ID NO: 21 consists essentially of amino acids 1 to 483 of SEQ ID NO: 21, the mature polypeptide of SEQ ID NO: 22 consists essentially of amino acids 1 to 483 of SEQ ID NO: 22, the mature polypeptide of SEQ ID NO: 23 consists essentially of amino acids 1 to 483 of SEQ ID NO: 23, the mature polypeptide of SEQ ID NO: 24 consists essentially of amino acids 1 to 483 of SEQ ID NO: 24, the mature polypeptide of SEQ ID NO: 25 consists essentially of amino acids 1 to 483 of SEQ ID NO: 25, the mature polypeptide of SEQ ID NO: 26 consists essentially of amino acids 21 to 607 of SEQ ID NO: 26, and/or the mature polypeptide of SEQ ID NO: 27 consists essentially of amino acids 19 to 640 of SEQ ID NO: 27.

In further embodiments, the mature polypeptide of SEQ ID NO: 2 consists of amino acids 18 to 573 of SEQ ID NO: 2, the mature polypeptide of SEQ ID NO: 3 consists of amino acids 18 to 573 of SEQ ID NO: 3, the mature polypeptide of SEQ ID NO: 4 consists of amino acids 18 to 573 of SEQ ID NO: 4, the mature polypeptide of SEQ ID NO: 5 consists of amino acids 18 to 573 of SEQ ID NO: 5, the mature polypeptide of SEQ ID NO: 6 consists of amino acids 18 to 573 of SEQ ID NO: 6, the mature polypeptide of SEQ ID NO: 7 consists of amino acids 18 to 573 of SEQ ID NO: 7, the mature polypeptide of SEQ ID NO: 8 consists of amino acids 18 to 573 of SEQ ID NO: 8, the mature polypeptide of SEQ ID NO: 9 consists of amino acids 18 to 576 of SEQ ID NO: 9, the mature polypeptide of SEQ ID NO: 10 consists of amino acids 18 to 576 of SEQ ID NO: 10, the mature polypeptide of SEQ ID NO: 11 consists of amino acids 21 to 618 of SEQ ID NO: 11, the mature polypeptide of SEQ ID NO: 12 consists of amino acids 1 to 559 of SEQ ID NO: 12, the mature polypeptide of SEQ ID NO: 13 consists of amino acids 1 to 928 of SEQ ID NO: 13, the mature polypeptide of SEQ ID NO: 14 consists essentially of amino acids 1 to 828 of SEQ ID NO: 14, the mature polypeptide of SEQ ID NO: 15 consists of amino acids 1 to 928 of SEQ ID NO: 15, the mature polypeptide of SEQ ID NO: 16 consists of amino acids 1 to 928 of SEQ ID NO: 16, the mature polypeptide of SEQ ID NO: 17 consists of amino acids 1 to 484 of SEQ ID NO: 17, the mature polypeptide of SEQ ID NO: 18 consists of amino acids 1 to 583 of SEQ ID NO: 18, the mature polypeptide of SEQ ID NO: 19 consists of amino acids 22 to 471 of SEQ ID NO: 19, the mature polypeptide of SEQ ID NO: 20 consists of amino acids 1 to 514 of SEQ ID NO: 20, the mature polypeptide of SEQ ID NO: 21 consists of amino acids 1 to 483 of SEQ ID NO: 21, the mature polypeptide of SEQ ID NO: 22 consists of amino acids 1 to 483 of SEQ ID NO: 22, the mature polypeptide of SEQ ID NO: 23 consists of amino acids 1 to 483 of SEQ ID NO: 23, the mature polypeptide of SEQ ID NO: 24 consists of amino acids 1 to 483 of SEQ ID NO: 24, the mature polypeptide of SEQ ID NO: 25 consists of amino acids 1 to 483 of SEQ ID NO: 25, the mature polypeptide of SEQ ID NO: 26 consists of amino acids 21 to 607 of SEQ ID NO: 26, the mature polypeptide of SEQ ID NO: 27 consists of amino acids 19 to 640 of SEQ ID NO: 27.

The prediction of mature polypeptide sequences may be based on the SignalP program (Nielsen et al., 1997, Protein Engineering 10: 1-6) that predicts amino acids 1 to 17 of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10 are a signal peptide. Likewise, amino acids 1 to 20 of SEQ ID NO: 11, amino acids 1 to 21 of SEQ ID NO: 19, amino acids 1 to 20 of SEQ ID NO: 26, and amino acids 1-18 of SEQ ID NO: 27 are predicted to be signal peptides.

The sequence defined by amino acids 19 to 474 (particularly 19 to 471) of SEQ ID NO: 2, SEQ ID NO: 3, SEQ ID NO: 4, SEQ ID NO: 5, SEQ ID NO: 6, SEQ ID NO: 7, or SEQ ID NO: 8 or amino acids 19 to 471 of SEQ ID NO: 9 or of SEQ ID NO: 10 is the catalytic domain. The sequence defined by amino acids 480 to 573 of SEQ ID NO: 2, SEQ ID NO: 3, SEQ ID NO: 4, SEQ ID NO: 5, SEQ ID NO: 6, SEQ ID NO: 7, or SEQ ID NO: 8 or amino acids 483 to 576 of SEQ ID NO: 9 or SEQ ID NO: 10 is a starch binding domain.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

Degree of polymerization (DP): DP refers to the number (n) of anhydroglucopyranose units in a given saccharide. Examples of DP1 are monosaccharides, such as glucose and fructose. DP2 are disaccharides, such as maltose and sucrose.

DS and % DS: As used herein, “DS” is an abbreviation of the term “Dry Solid”. In the context of the present invention the solid content of liquefied starch (including starch slurry or partially hydrolysed starch) is provided as percentage of dry solid (% DS) unless otherwise stated. The percentage of dry solid is calculated based on weight (w/w %).

% DX: In the context of the present invention dextrose percentage (% DX) is used as a measure of the amounts of dextrose obtained during saccharification or after completion of the saccharification process. The dextrose percentage is calculated based on weight (w/w %).

### Aspects and Embodiments of the Invention

The present inventors have surprisingly found that delayed or staged addition of alpha-amylase during saccharification of liquefied starch has several benefits: the positive effect of the alpha-amylase on saccharification speed is maintained and, in addition, the peak % DX is much improved. This beneficial effect is observed even when the alpha-amylase is added at a relatively late stage in the saccharification process. In addition, the inventors have found that increasing the ratio of pullulanase to glucoamylase in the enzyme blend added at onset of the saccharification process also aids in arriving at a higher % DX in the saccharification product.

In a first aspect the present invention provides a composition comprising a glucoamylase and a pullulanase, wherein the ratio of pullulanase activity expressed as NPUN/g to glucoamylase activity expressed as AGU/g is higher than 6, such as higher than 7, higher than 8, higher than 9 or within the range of 6-20, such as within the range of 6-19, within the range of 6-18. within the range of 6-17, within the range of 6-16, within the range of 6-15, within the range of 6-14, within the range of 6-13, within the range of 6-12, within the range of 6-11, within the range of 7-20, within the range of 7-19, within the range of 7-18, within the range of 7-17, within the range of 7-16, within the range of 7-15, within the range of 7-14, within the range of 7-13, within the range of 7-12, within the range of 7-11, within the range of 8-20, within the range of 8-19, within the range of 8-18, within the range of 8-17, within the range of 8-16, within the range of 8-15, within the range of 8-14, within the range of 8-13, within the range of 8-12, within the range of 8-11, within the range of 9-20, within the range of 9-19, within the range of 9-18, within the range of 9-17, within the range of 9-16, within the range of 9-15, within the range of 9-14, within the range of 9-13, within the range of 9-12 or such as within the range of 9-11.

It is to be understood that when delaying the addition of alpha amylase during saccharification, it is desirable that the composition comprising glucoamylase and pullulanase which is added at onset of the saccharification contains no or at least very little alpha-amylase activity: Hence, in some embodiments of the invention the composition comprises no alpha-amylase activity. Alternatively, if said composition does comprise a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 29, such as higher than 50, higher than 100, higher than 200, higher than 300, higher than 400, higher than 500, higher than 600, higher than 800, higher than 1000, higher than 2000, higher than 3000, higher than 4000 or such as higher than 5000. If the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 29, such as higher than 50, higher than 100, higher than 200, higher than 300, higher than 400, higher than 500, higher than 600, higher than 800, higher than 1000, higher than 2000, higher than 3000, higher than 4000 or such as higher than 5000.

In some specific embodiments, the invention provides a composition, wherein the ratio of pullulanase activity expressed as NPUN/g to glucoamylase activity expressed as AGU/g is higher than 6, and wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 100, If the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 100.

In other specific embodiments, the invention provides a composition, wherein the ratio of pullulanase activity expressed as NPUN/g to glucoamylase activity expressed as AGU/g is within the range of 6-20, and wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 100, If the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 100.

In further specific embodiments, the invention provides a composition, wherein the ratio of pullulanase activity expressed as NPUN/g to glucoamylase activity expressed as AGU/g is within the range of 6-20, and wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 500. If the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 500.

In even further specific embodiments, the invention provides a composition, wherein the ratio of pullulanase activity expressed as NPUN/g to glucoamylase activity expressed as AGU/g is within the range of 7-15, and wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 100. If the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 100.

Other specific embodiments of the invention provides a composition, wherein the ratio of pullulanase activity expressed as NPUN/g to glucoamylase activity expressed as AGU/g is within the range of 7-15, and wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 500. If the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 500.

A glucoamylase included in the compositions or used in the processes according to the present invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al., 1984, EMBO J. 3 (5): 1097-1102), or variants thereof, such as those disclosed in WO 92/00381, WO 00/04136 and WO 01/04273 (from Novozymes, Denmark); the A. awamori glucoamylase disclosed in WO 84/02921, A. oryzae glucoamylase (Agric. Biol. Chem., 1991, 55 (4): 941-949), or variants or fragments thereof. Other Aspergillus glucoamylase variants include variants with enhanced thermal stability: G137A and G139A (Chen et al., 1996, Prot. Eng. 9: 499-505); D257E and D293E/Q (Chen et al., 1995, Prot. Eng. 8: 575-582); N182 (Chen et al., 1994, Biochem. J. 301: 275-281); disulphide bonds, A246C (Fierobe et al., 1996, Biochemistry 35: 8698-8704; and introduction of Pro residues in position A435 and S436 (Li et al., 1997, Protein Eng. 10: 1199-1204.

Other glucoamylases include Athelia rolfsii (previously denoted Corticium rolfsii) glucoamylase (see U.S. Pat. No. 4,727,026 and Nagasaka et al., 1998, “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, and Talaromyces thermophilus (U.S. Pat. No. 4,587,215).

Contemplated fungal glucoamylases include Trametes cingulata, disclosed in WO 2006/069289.

In an embodiment, the glucoamylase is derived from a strain of the genus Pycnoporus, in particular a strain of Pycnoporus as described in WO 2011/066576 (SEQ ID NOs 2, 4 or 6), or from a strain of the genus Gloeophyllum, in particular a strain of Gloeophyllum as described in WO 2011/068803 (SEQ ID NO: 2, 4, 6, 8, 10, 12, 14 or 16) or a strain of the genus Nigrofomes, in particular a strain of Nigrofomes sp. disclosed in WO 2012/064351 (SEQ ID NO: 2) (all references hereby incorporated by reference) or a strain of Penicillium, in particular Penicillium oxalicum disclosed in WO2011/127802 (SEQ ID NO: 2) or WO2013/036526. Contemplated are also glucoamylases which exhibit a high identity to any of the above-mentioned glucoamylases, i.e., at least 70%, at least 75%, at least 80%, at least 85%, at least 90%, at least 95%, at least 96%, at least 97%, at least 98%, at least 99%, such as 100% identity to any one of the mature parts of the enzyme sequences mentioned above.

In an embodiment, the glucoamylase is derived from a strain of the genus Trichoderma, in particular as described in WO2009/048487, WO2009/048488, WO2008/045489, WO2011/022465, WO2012/001139.

Commercially available glucoamylase compositions include AMG 200L; AMG 300L; SANT™ SUPER, SANT™ EXTRA L, SPIRIZYME™ PLUS, SPIRIZYME™ FUEL, SPIRIZYME™ B4U, SPIRIZYME ULTRA™, SPIRIZYME EXCEL™ and AMG™ E (from Novozymes NS, Denmark); OPTIDEX™ 300, GC480™ and GC147™ (from Genencor Int., USA); AMIGASE™ and AMIGASE™ PLUS (from DSM); G-ZYME™ G900, G-ZYME™ and G990 ZR (from DuPont-Genencor)

According to specific embodiments, the said glucoamylase is selected from the group consisting of an Aspergillus niger glucoamylase, a Talaromyces emersonii glucoamylase, a Trametes cingulata glucoamylase a Gloeophyllum trabeum glucoamylase, a Trichoderma Reesei glucoamylase, a Humicola insolens glucoamylase and an Aspergillus fumigatus glucoamylase, and hybrids and variants thereof.

In principle, any pullulanase may be used in a process of the present invention. In an embodiment, the pullulanase is a pullulanase from Bacillus deramificans, e.g., disclosed in U.S. Pat. No. 6,074,854 and U.S. Pat. No. 5,817,498, or a pullulanase derived from Bacillus acidopullulyticus, e.g., disclosed in WO2009/075682 (SEQ ID 4; GENESEQP: AXB71624). The pullulanase may also be a hybrid or a variant of any of these pullulanases.

Commercially available pullulanases include Promozyme D2 available from Novozymes NS, Bagsvaerd, Denmark), Novozym 26062 (Novozymes) and Optimax L 1000 (DuPont-Genencor).

The composition according to any of the preceding claims, wherein the pullulanase comprises/consists essentially of/consists of an amino acid sequence selected from the group consisting of
i) The amino acid sequence set forth in any one of SEQ ID NOs: 13-16 or a mature polypeptide thereof;
ii) A subsequence of the amino acid sequence set forth in any one of SEQ ID NO: 13-16 or of said mature polypeptide thereof;
iii) A variant amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii);

wherein when said pullulanase comprises a subsequence as defined in ii) or a variant amino acid sequence as defined in iii), it preferably has at least 80%, such as at least 90% of the pullulanase activity of the respective amino acid defined in i) of which it is a subsequence or variant, when tested as set forth above in relation to the definition of “Pullulanase activity (NPUN)”.

In the composition according to the invention, the glucoamylase may in particular comprise an amino acid sequence selected from the group consisting of:
i) The amino acid sequence set forth in any one of SEQ ID NOs: 2-12 and 27 or a mature polypeptide thereof;
ii) A subsequence of the amino acid sequence set forth in any one of SEQ ID NOs: 2-12 and 27 or of said mature polypeptide thereof;
iii) A variant amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii)

wherein when said glucoamylase comprises a subsequence as defined in ii) or an amino acid sequence as defined in iii), it preferably has at least 80%, such as at least 90% of the glucoamylase activity of the respective amino acid defined in i) of which it is a subsequence or variant, when tested as set forth above in relation to the definition of “Glucoamylase activity (AGU)”.

In particular, the composition according to the invention may comprise:
i) a glucoamylase comprising an amino acid sequence selected from the group consisting of the amino acid sequence set forth in SEQ ID NO: 12 or a mature polypeptide thereof,
ii) a subsequence of the amino acid sequence set forth in SEQ ID NO: 12 or of said mature polypeptide thereof, and
iii) an amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii);
wherein when said glucoamylase is a subsequence as defined in ii) or a variant amino acid sequence as defined in iii), it preferably has at least 80%, such as at least 90% of the glucoamylase activity of the respective amino acid defined in i) of which it is a subsequence or variant, when tested as set forth above in relation to the definition of “Glucoamylase activity (AGU)”.

In combination with
iv) a pullulanase comprising an amino acid sequence selected from the group consisting of the amino acid sequence set forth in any one of SEQ ID NOs: 13-16 or a mature polypeptide thereof,
v) a subsequence of the amino acid sequence set forth in any one of SEQ ID NOs: 13-16 or of said mature polypeptide thereof, and
vi) an amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in iv) and v);
wherein when said pullulanase comprises is a subsequence as defined in v) or a variant amino acid sequence as defined in vi), it preferably has at least 80%, such as at least 90% of the pullulanase activity of the respective amino acid defined in iv) of which it is a subsequence or variant, when tested as set forth above in relation to the definition of “Pullulanase activity (NPUN)”.

In related embodiments, the glucoamylase consists essentially of an amino acid sequence as set forth in any of items i)-iii) above. In other related embodiments/consists the glucoamylase consists of an amino acid sequence as set forth in any of items i)-iii) above.

In the composition according to any of the preceding claims, the alpha-amylase may comprise an amino acid sequence selected from the group consisting of
i) The amino acid sequence set forth in any one of SEQ ID NOs: 1 and 17-26 or a mature polypeptide thereof;
ii) A subsequence of the amino acid sequence set forth in any one of SEQ ID NOs: 1 and 17-26 or of said mature polypeptide thereof;
iii) An amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii).

When the alpha-amylase defined above is a subsequence or a variant, it preferably has at least 80%, such as at least 90% of the alpha-amylase activity of the respective alpha-amylase selected from SEQ ID NOs: 1 and 17-26 or the mature polypeptide thereof of which it is a subsequence or variant, when tested as set forth above in relation to the definition of “Acid alpha-Amylase Units (FAU(A))” or “Alpha-amylase Activity (KNU)”: In the present context an alpha-amylase comprising an amino acid sequences selected from the group consisting of SEQ ID NOs: 1, 17-19 and 26 or the mature polypeptide thereof, is considered to be a fungal alpha amylase and activity is tested as provided in relation to the above definition of “Acid alphaAmylase Units (FAU(A))”. An alpha-amylase comprising an amino acid sequence selected from the group consisting of SEQ ID NOs: 20-25 or the mature polypeptide thereof is considered to be a bacterial alpha amylase and activity is tested as provided in relation to the above definition of “Kilo Novo alpha-amylase Units (KNU)”.

In related embodiments, the alpha-amylase consists essentially of an amino acid sequence as set forth in any of items i)-iii) above. In other related embodiments the alpha-amylase consists of an amino acid sequence as set forth in any of items i)-iii) above.

The composition provided according to the invention may be a liquid or solid composition. In currently preferred embodiments it is a liquid composition.

Another aspect of the invention provides a process for producing glucose syrup. In the process according to the invention the addition of alpha amylase is staged in the sense that glucoamylase and pullulanase is added at onset of the saccharification reaction, while the addition of alpha-amylase is postponed at least until a significant or substantial part of the liquefied starch has been saccharified. Hence, the process of the invention comprises,
i) providing a composition comprising liquefied starch, and adding to said composition a glucoamylase (AMG) and a pullulanase;
ii) subjecting the composition to incubation under conditions allowing starch hydrolysis/saccharification, and subsequently
iii) adding an alpha-amylase to the said composition and subjecting the composition to incubation under conditions allowing starch hydrolysis/saccharification.

The amounts of glucoamylase added in step i) of the process may in particular correspond to 0.05-0.5 AGU/gDS, such as 0.05-0.4 AGU/gDS, 0.05-0.35 AGU/gDS, 0.05-0.3 AGU/gDS, 0.075-0.5 AGU/gDS, 0.075-0.4 AGU/gDS, 0.075-0.35 AGU/gDS 0.075-0.3 AGU/gDS, 0.075-0.5 AGU/gDS 0.1-0.5 AGU/gDS, 0.1-0.4 AGU/gDS, 0.1-0.35 AGU/gDS, 0.1-0.3 AGU/gDS, 0.125-0.5 AGU/gDS, 0.125-0.4 AGU/gDS, 0.125-0.35 AGU/gDS, 0.125-0.3 AGU/gDS, 0.15-0.5 AGU/gDS, 0.15-0.4 AGU/gDS, 0.15-0.35 AGU/gDS, 0.15-0.3 AGU/gDS, 0.175-0.5 AGU/gDS, 0.175-0.4 AGU/gDS, 0.175-0.35 AGU/gDS, 0.175-0.3 AGU/gDS, 0.2-0.5 AGU/gDS, 0.2-0.4 AGU/gDS, 0.2-0.35 AGU/gDS, 0.2-0.3 AGU/gDS, 0.24-0.5 AGU/gDS, 0.24-0.4 AGU/gDS, 0.24-0.35 AGU/gDS or such as 0.24-0.26 AGU/gDS.

The amounts of pullulanase added in step i) of the process may in particular correspond to 0.05-5 NPUN(X)/gDS, such as 0.05-4.5 NPUN(X)/gDS, 0.05-4 NPUN(X)/gDS, 0.05-3.5 NPUN(X)/gDS, 0.05-3 NPUN(X)/gDS, 0.05-2.5 NPUN(X)/gDS, 0.1-5 NPUN(X)/gDS, 0.1-4 NPUN(X)/gDS, 0.1-3.5 NPUN(X)/gDS, 0.1-3 NPUN(X)/gDS, 0.1-2.5 NPUN(X)/gDS, 0.25-5 NPUN(X)/gDS, 0.25-4.5 NPUN(X)/gDS, 0.25-4 NPUN(X)/gDS, 0.25-3.5 NPUN(X)/gDS, 0.25-3 NPUN(X)/gDS, 0.5-5 NPUN(X)/gDS, 0.5-4.5 NPUN(X)/gDS, 0.5-4 NPUN(X)/gDS, 0.5-3.5 NPUN(X)/gDS, 0.05-3 NPUN(X)/gDS, 0.5-2.5 NPUN(X)/gDS, 1.0-5 NPUN(X)/gDS, 1.0-4.5 NPUN(X)/gDS, 1.0-4 NPUN(X)/gDS, 1.0-3.5 NPUN(X)/gDS, 1.0-3 NPUN(X)/gDS, 1.0-2.5 NPUN(X)/gDS, 1.5-5 NPUN(X)/gDS, 1.5-4.5 NPUN(X)/gDS, 1.5-4 NPUN(X)/gDS, 1.5-3.5 NPUN(X)/gDS, 1.5-3 NPUN(X)/gDS, 1.5-2.5 NPUN(X)/gDS, 2-5 NPUN(X)/gDS, 2-4.5 NPUN(X)/gDS, 2-4 NPUN(X)/gDS, 2-3.5 NPUN(X)/gDS, 2-3 NPUN(X)/gDS, 2-5 NPUN(X)/gDS, 2-2.5 NPUN(X)/gDS, 2.25-5 NPUN(X)/gDS, 2.25-4.5 NPUN(X)/gDS, 2.25-4 NPUN(X)/gDS, 2.25-3.5 NPUN(X)/gDS, 2.25-3 NPUN(X)/gDS, 2.25-2.5 NPUN(X)/gDS, or such as 2.50-2.54 NPUN(X)/gDS.

Where the alpha-amylase added in step iii) of the process according to the invention is a fungal alpha-amylase, the amounts may in particular correspond to 0.0005-0.025 FAU(A)/gDS, such as 0.0005-0.0225 FAU(A)/gDS, 0.0005-0.02 FAU(A)/gDS, 0.0005-0.0175 FAU(A)/gDS, 0.0005-0.015 FAU(A)/gDS, 0.0005-0.0125 FAU(A)/gDS, 0.001-0.025 FAU(A)/gDS, 0.001-0.0225 FAU(A)/gDS, 0.001-0.02 FAU(A)/gDS, 0.001-0.0175 FAU(A)/gDS, 0.001-0.015 FAU(A)/gDS, 0.001-0.0125 FAU(A)/gDS, 0.0025-0.025 FAU(A)/gDS, 0.0025-0.0225 FAU(A)/gDS, 0.0025-0.02 FAU(A)/gDS, 0.0025-0.0175 FAU(A)/gDS, 0.0025-0.015 FAU(A)/gDS, 0.0025-0.0125 FAU(A)/gDS, 0.005-0.025 FAU(A)/gDS, 0.005-0.0225 FAU(A)/gDS, 0.005-0.02 FAU(A)/gDS, 0.005-0.0175 FAU(A)/gDS, 0.005-0.015 FAU(A)/gDS, 0.005-0.0125 FAU(A)/gDS, 0.0075-0.025 FAU(A)/gDS, 0.0075-0.0225 FAU(A)/gDS, 0.0075-0.02 FAU(A)/gDS, 0.0075-0.0175 FAU(A)/gDS, 0.0075-0.015 FAU(A)/gDS, 0.0075-0.0125 FAU(A)/gDS, 0.008-0.025 FAU(A)/gDS, 0.008-0.0225 FAU(A)/gDS, 0.008-0.02 FAU(A)/gDS, 0.008-0.0175 FAU(A)/gDS, 0.008-0.015 FAU(A)/gDS, 0.008-0.0125 FAU(A)/gDS, 0.009-0.01 FAU(A)/gDS or such as 0.0095 FAU(A)/gDS.

Where the alpha-amylase added in step iii) of the process according to the invention is a bacterial alpha-amylase, the amounts may in particular correspond to 0.0005-0.025 KNU/gDS, such as 0.0005-0.0225 KNU/gDS, 0.0005-0.02 KNU/gDS, 0.0005-0.0175 KNU/gDS, 0.0005-0.015 KNU/gDS, 0.0005-0.0125 KNU/gDS, 0.001-0.025 KNU/gDS, 0.001-0.0225 KNU/gDS, 0.001-0.02 KNU/gDS, 0.001-0.0175 KNU/gDS, 0.001-0.015 KNU/gDS, 0.001-0.0125 KNU/gDS, 0.0025-0.025 KNU/gDS, 0.0025-0.0225 KNU/gDS, 0.0025-0.02 KNU/gDS, 0.0025-0.0175 KNU/gDS, 0.0025-0.015 KNU/gDS, 0.0025-0.0125 KNU/gDS, 0.005-0.025 KNU/gDS, 0.005-0.0225 KNU/gDS, 0.005-0.02 KNU/gDS, 0.005-0.0175 KNU/gDS, 0.005-0.015 KNU/gDS, 0.005-0.0125 KNU/gDS, 0.0075-0.025 KNU/gDS, 0.0075-0.0225 KNU/gDS, 0.0075-0.02 KNU/gDS, 0.0075-0.0175 KNU/gDS, 0.0075-0.015 KNU/gDS, 0.0075-0.0125 KNU/gDS, 0.008-0.025 KNU/gDS, 0.008-0.0225 KNU/gDS, 0.008-0.02 KNU/gDS, 0.008-0.0175 KNU/gDS, 0.008-0.015 KNU/gDS, 0.008-0.0125 KNU/gDS, 0.009-0.01 KNU/gDS or such as 0.0095 KNU/gDS.

In some embodiments, the invention provides a process, wherein the amounts of glucoamylase added in step i) of the process correspond to 0.05-0.5 AGU/gDS, the amounts of pullulanase added in step i) of the process correspond to 0.05-5 NPUN(X)/gDS, and the amounts of fungal alpha-amylase added in step iii) of the process correspond to 0.0005-0.025 (FAU)(A)/gDS or the amounts of bacterial alpha-amylase added in step iii) of the process correspond to 0.0005-0.025 KNU/gDS.

In still other embodiments, the invention provides a process, wherein the amounts of glucoamylase added in step i) of the process correspond to 0.175-0.3 AGU/gDS, the amounts of pullulanase added in step i) of the process correspond to 2-4 NPUN(X)/gDS, and the amounts of alpha-amylase added in step iii) of the process correspond to 0.0025-0.02 (FAU)(A)/gDS or the amounts of bacterial alpha-amylase added in step iii) of the process correspond to 0.0025-0.02 KNU/gDS.

In further embodiments, the invention pertains to a process, wherein the amounts of glucoamylase added in step i) of the process correspond to 0.2-0.3 AGU/gDS, the amounts of pullulanase added in step i) of the process correspond to 2-3 NPUN(X)/gDS, and the amounts of alpha-amylase added in step iii) of the process correspond to 0.008-0.0125 (FAU)(A)/gDS or the amounts of bacterial alpha-amylase added in step iii) of the process correspond to 0.008-0.0125 KNU/gDS.

When delaying the addition of alpha amylase during saccharification, it is desirable that no or at least very little alpha-amylase activity is added together with the glucoamylase and the pullulanase at onset of the saccharification process. Therefore, in some embodiments the process comprising adding to said composition comprising liquefied starch a formulation comprising a glucoamylase and a pullulanase, wherein if said formulation comprises a polypeptide having alpha-amylase activity, and that polypeptide is of bacterial origing, then the ratio of glucoamylase activity expressed as AGU/g to alpha-amylase activity expressed as Fungal alphaAmylase Units FAU(A)/g in said composition is higher than 29, such as higher than 50, higher than 100, higher than 200, higher than 300, higher than 400, higher than 500, higher than 600, higher than 800, higher than 1000, higher than 2000, higher than 3000, higher than 4000 or such as higher than 5000. If the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 29, such as higher than 50, higher than 100, higher than 200, higher than 300, higher than 400, higher than 500, higher than 600, higher than 800, higher than 1000, higher than 2000, higher than 3000, higher than 4000 or such as higher than 5000.

According to some embodiments, the glucoamylase and the pullulanase added in step ii) of the process are in a composition according to the invention as defined in the above.

The incubation in step ii) of the process may have a duration of from 2-35 hours, such as from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-35 hours, from 10-30 hours, from 10-28 hours, from 10-26 hours, from 10-24 hours, from 10-22 hours, from 10-20 hours, from 10-18 hours, from 10-16 hours, from 10-14 hours, from 10-12 hours, from 14-35 hours, from 14-30 hours, from 14-28 hours, from 14-26 hours, from 14-24, from 14-22 hours, from 14-20 hours, from 14-18 hours, from 14-16 hours, from 18-35 hours, from 18-30 hours, from 18-28 hours, from 18-26 hours, from 18-24, from 18-22 hours, from 18-20 hours, from 20-35 hours, from 20-30 hours, from 20-28 hours, from 20-26 hours, from 20-24, from 20-22 hours, form 22-35 hours, from 22-30 hours, from 22-28 hours, from 22-26 hours, from 22-24 hours or such or sus as a duration of 24 hours.

The incubation in step iii) of the process may have a duration of from 2-94 hours, such as from 2-90 hours, from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-94 hours, from 4-90 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-94 hours, from 10-90 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-94 hours, from 14-90 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-94 hours, from 20-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-94 hours, from 24-90 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-94 hours, from 30-90 hours, from 30-80 hours, from 30-70 hours, from 30-60, from 30-55 hours, from 30-50 hours, from 30-45 hours, from 30-40 hours, from 30-35 hours, from 40-94 hours, from 40-90 hours, from 40-80 hours, from 40-70 hours, from 40-60, from 40-55 hours, from 40-50 hours, from 40-45 hours, from 50-94 hours, from 50-90 hours, from 50-80 hours, from 50-70 hours, from 50-60, from 60-94 hours, from 60-90 hours, from 60-80 hours, or such as a duration of from 60-70 hours.

In specific embodiments, the incubation in step ii) of the process has a duration of from 2-35 hours and said incubation in step iii) of the process has a duration of from 2-94 hours, such as from 2-90 hours, from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-94 hours, from 4-90 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-94 hours, from 10-90 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-94 hours, from 14-90 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-94 hours, from 20-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-94 hours, from 24-90 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-94 hours, from 30-90 hours, from 30-80 hours, from 30-70 hours, from 30-60, from 30-55 hours, from 30-50 hours, from 30-45 hours, from 30-40 hours, from 30-35 hours, from 40-94 hours, from 40-90 hours, from 40-80 hours, from 40-70 hours, from 40-60, from 40-55 hours, from 40-50 hours, from 40-45 hours, from 50-94 hours, from 50-90 hours, from 50-80 hours, from 50-70 hours, from 50-60, from 60-94 hours, from 60-90 hours, from 60-80 hours, or such as a duration of from 60-70 hours.

In other specific embodiments, the said incubation in step ii) of the process has a duration of from 2-20 hours and said incubation in step iii) of the process has a duration of from 2-94 hours, such as from 2-90 hours, from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-94 hours, from 4-90 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-94 hours, from 10-90 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-94 hours, from 14-90 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-94 hours, from 20-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-94 hours, from 24-90 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-94 hours, from 30-90 hours, from 30-80 hours, from 30-70 hours, from 30-60, from 30-55 hours, from 30-50 hours, from 30-45 hours, from 30-40 hours, from 30-35 hours, from 40-94 hours, from 40-90 hours, from 40-80 hours, from 40-70 hours, from 40-60, from 40-55 hours, from 40-50 hours, from 40-45 hours, from 50-94 hours, from 50-90 hours, from 50-80 hours, from 50-70 hours, from 50-60, from 60-94 hours, from 60-90 hours, from 60-80 hours, or such as a duration of from 60-70 hours.

In still other embodiments, the said incubation in step ii) of the process has a duration of from 4-35 hours and said incubation in step iii) of the process has a duration of from 2-92 hours, such as from 2-90 hours, from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-92 hours, from 4-90 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-92 hours, from 10-90 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-92 hours, from 14-90 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-92 hours, from 20-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-92 hours, from 24-90 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-92 hours, from 30-90 hours, from 30-80 hours, from 30-70 hours, from 30-60, from 30-55 hours, from 30-50 hours, from 30-45 hours, from 30-40 hours, from 30-35 hours, from 40-92 hours, from 40-90 hours, from 40-80 hours, from 40-70 hours, from 40-60, from 40-55 hours, from 40-50 hours, from 40-45 hours, from 50-92 hours, from 50-90 hours, from 50-80 hours, from 50-70 hours, from 50-60, from 60-94 hours, from 60-90 hours, from 60-80 hours, or such as a duration of from 60-70 hours.

In further embodiments, the said incubation in step ii) of the process has a duration of from 4-22 hours, and said incubation in step iii) of the process has a duration of from 2-92 hours, such as from 2-90 hours, from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-92 hours, from 4-90 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-92 hours, from 10-90 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-92 hours, from 14-90 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-92 hours, from 20-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-92 hours, from 24-90 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-92 hours, from 30-90 hours, from 30-80 hours, from 30-70 hours, from 30-60, from 30-55 hours, from 30-50 hours, from 30-45 hours, from 30-40 hours, from 30-35 hours, from 40-92 hours, from 40-90 hours, from 40-80 hours, from 40-70 hours, from 40-60, from 40-55 hours, from 40-50 hours, from 40-45 hours, from 50-92 hours, from 50-90 hours, from 50-80 hours, from 50-70 hours, from 50-60, from 60-94 hours, from 60-90 hours, from 60-80 hours, or such as a duration of from 60-70 hours.

In still further embodiments, the said incubation in step ii) of the process has a duration of from 10-35 hours, and said incubation in step iii) of the process has a duration of from 2-86 hours, such as from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-86 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-86 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-86 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-86 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-86 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-86 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-86 hours, from 30-80 hours, from 30-70 hours, from 30-60, from 30-55 hours, from 30-50 hours, from 30-45 hours, from 30-40 hours, from 30-35 hours, from 40-86 hours, from 40-80 hours, from 40-70 hours, from 40-60, from 40-55 hours, from 40-50 hours, from 40-45 hours, from 50-86 hours, from 50-80 hours, from 50-70 hours, from 50-60, from 60-94 hours, from 60-90 hours, from 60-80 hours, or such as a duration of from 60-70 hours.

In even further embodiments, the said incubation in step ii) of the process has a duration of from 10-24 hours, and said incubation in step iii) of the process has a duration of from 2-86 hours, such as from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-86 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-86 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-86 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-86 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-86 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-86 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-86 hours, from 30-80 hours, from 30-70 hours, from 30-60, from 30-55 hours, from 30-50 hours, from 30-45 hours, from 30-40 hours, from 30-35 hours, from 40-86 hours, from 40-80 hours, from 40-70 hours, from 40-60, from 40-55 hours, from 40-50 hours, from 40-45 hours, from 50-86 hours, from 50-80 hours, from 50-70 hours, from 50-60, from 60-94 hours, from 60-90 hours, from 60-80 hours, or such as a duration of from 60-70 hours.

In yet further embodiments, the said incubation in step ii) of the process has a duration of from 14-35 hours, and said incubation in step iii) of the process has a duration of from 2-70 hours, such as from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-94 hours, from 25-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-70 hours, from 30-60, from 30-55 hours, from 30-50 hours, from 30-45 hours, from 30-40 hours, from 30-35 hours, from 40-70 hours, from 40-60, from 40-55 hours, from 40-50 hours, from 40-45 hours, from 50-70 hours, from 50-60, from 60-94 hours, from 60-90 hours, from 60-80 hours, or such as a duration of from 60-70 hours.

In even further embodiments, the incubation in step ii) of the process has a duration of from 14-26 hours, and said incubation in step iii) of the process has a duration of from 2-70 hours, such as from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-94 hours, from 25-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-70 hours, from 30-60, from 30-55 hours, from 30-50 hours, from 30-45 hours, from 30-40 hours, from 30-35 hours, from 40-70 hours, from 40-60, from 40-55 hours, from 40-50 hours, from 40-45 hours, from 50-70 hours, from 50-60, from 60-94 hours, from 60-90 hours, from 60-80 hours, or such as a duration of from 60-70 hours.

In other embodiments, the said incubation in step ii) of the process has a duration of from 18-35 hours, and said incubation in step iii) of the process has a duration of from 2-78 hours, such as from 2-74 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-78 hours, from 4-74 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-76 hours, from 10-74 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-78 hours, from 14-74 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-78 hours, from 20-74 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-78 hours, from 24-76 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-78 hours, from 30-74 hours, from 30-70 hours, from 30-60, from 30-55 hours, from 30-50 hours, from 30-45 hours, from 30-40 hours, from 30-35 hours, from 40-76 hours, from 40-74 hours, from 40-70 hours, from 40-60, from 40-55 hours, from 40-50 hours, from 40-45 hours, from 50-78 hours, from 50-74 hours, from 50-70 hours, from 50-60, from 60-94 hours, from 60-90 hours, from 60-80 hours, or such as a duration of from 60-70 hours.

In further embodiments, the said incubation in step ii) of the process has a duration of from 18-28 hours, and said incubation in step iii) of the process has a duration of from 2-78 hours, such as from 2-74 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-78 hours, from 4-74 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-76 hours, from 10-74 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-78 hours, from 14-74 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-78 hours, from 20-74 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-78 hours, from 24-76 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-78 hours, from 30-74 hours, from 30-70 hours, from 30-60, from 30-55 hours, from 30-50 hours, from 30-45 hours, from 30-40 hours, from 30-35 hours, from 40-76 hours, from 40-74 hours, from 40-70 hours, from 40-60, from 40-55 hours, from 40-50 hours, from 40-45 hours, from 50-78 hours, from 50-74 hours, from 50-70 hours, from 50-60, from 60-94 hours, from 60-90 hours, from 60-80 hours, or such as a duration of from 60-70 hours.

In even further embodiments, the said incubation in step ii) of the process has a duration of from 20-35 hours, and said incubation in step iii) of the process has a duration of from 2-76 hours, such as from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-76 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-76 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-76 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-76 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-76 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-76 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-76 hours, from 30-70 hours, from 30-60, from 30-55 hours, from 30-50 hours, from 30-45 hours, from 30-40 hours, from 30-35 hours, from 40-76 hours, from 40-70 hours, from 40-60, from 40-55 hours, from 40-50 hours, from 40-45 hours, from 50-76 hours, from 50-70 hours, from 50-60, from 60-94 hours, from 60-90 hours, from 60-80 hours, or such as a duration of from 60-70 hours.

In still further embodiments, the said incubation in step ii) of the process has a duration of from 20-30 hours, and said incubation in step iii) of the process has a duration of from 2-76 hours, such as from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-76 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-76 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-76 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-76 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-76 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-76 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-76 hours, from 30-70 hours, from 30-60, from 30-55 hours, from 30-50 hours, from 30-45 hours, from 30-40 hours, from 30-35 hours, from 40-76 hours, from 40-70 hours, from 40-60, from 40-55 hours, from 40-50 hours, from 40-45 hours, from 50-76 hours, from 50-70 hours, from 50-60, from 60-94 hours, from 60-90 hours, from 60-80 hours, or such as a duration of from 60-70 hours.

In even further embodiments, the said incubation in step ii) of the process has a duration of from 22-35 hours, and said incubation in step iii) of the process has a duration of from 2-74 hours, such as from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-74 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-74 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-74 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-74 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-94 hours, from 25-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-74 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-94 hours, from 30-90 hours, from 30-80 hours, from 30-70 hours, from 30-60, from 30-55 hours, from 30-50 hours, from 30-45 hours, from 30-40 hours, from 30-35 hours, from 40-74 hours, from 40-70 hours, from 40-60, from 40-55 hours, from 40-50 hours, from 40-45 hours, from 50-94 hours, from 50-90 hours, from 50-80 hours, from 50-70 hours, from 50-60, from 60-74 hours, or such as a duration of from 60-70 hours.

The incubation in step iii) of claim 14 may be allowed to proceed until the amount of glucose (% DX) in the composition has reached a level corresponding to 95% (w/w) or more, such as 95.5% (w/w) or more, 95.75% (w/w) or more, 96% (w/w) or more, 96.25% (w/w) or more, or such as 96.75% (w/w) or more.

In some embodiments, the duration of the process from addition of said a glucoamylase (AMG) and said pullulanase to the composition comprising liquefied starch until termination of the starch hydrolysis/saccharification is from 24-96 hours, such as from 24-72 hours, from 24-64 hours, from 24-48 hours, from 24-36 hours, from 24-32 hours, from 32-96 hours, from 32-72 hours, from 32-64 hours, from 32-48 hours, from 32-36 hours, from 36-96 hours, from 36-72 hours, from 36-64 hours, from 36-48 hours, from 48-96 hours, such as from 48-72 hours, from 48-64 hours, from 56-96 hours, such as from 56-72 hours, or such as from 56-64 hours,

In the process according to the invention, the starch hydrolysis/saccharification may in particular take place at a pH which is within the range of 3.5-5.0, such as at pH in the range of 4.0-4.5, and at a temperature, which is within the range of 59-70° C., such as in the range of 59-65° C. or such as in the range of 59-62° C.

The liquefied starch used as substrate for the saccharification process according to the invention may be a starch slurry or partially hydrolysed starch (liquefact or maltodextrin). In particular, the in starch slurry or partly hydrolysed starch may have a Dextrose equivalent (DE) in the range of 5-42, such as in the range of 5-30, in the range of 8-18 or such as in the range of 9-14.

The starch may be from any source, in particular from corn, wheat or tapioca. The starch slurry or partially hydrolysed starch may have residual alpha amylase activity from the liquefaction process present or it may have been deactivated, such as by reducing the pH to below 4.5, while maintaining a high temperature (above 95° C.), to inactivate the liquefying alpha-amylase.

The conductivity of said starch slurry or partially hydrolysed starch may in particular be within the range of 0-500 microS/cm. According to some embodiments the calcium content corresponds to 0-200 ppm free calcium.

In the process according to the invention, the starch hydrolysis/saccharification may in particular take place at a pH which is within the range of 3.5-5.0, such as at pH in the range of 4.0-4.7, and at a temperature, which is within the range of 58−70° C., such as in the range of 58-65° C., in the range of 59−65° C. or such as in the range of 59−62° C.

The composition comprising liquefied starch provided as a starting material; i.e. composition comprising liquefied starch provided the in step i) of the process may contain from 25-45% dry solids (% DS), such as from 25-40% DS, from 30-38% DS, from 32-38% DS, or such as from 34-36% DS.

Fungal alpha-amylases useful in the processes according to the invention include alpha-amylases derived from a strain of the genus Aspergillus, such as, Aspergillus oryzae, Aspergillus niger and Aspergillus kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e., more than 70%, more than 75%, more than 80%, more than 85% more than 90%, more than 95%, more than 96%, more than 97%, more than 98%, more than 99% or even 100% identity to the mature part of the amino acid sequence shown in SEQ ID NO: 10 in WO 96/23874.

Another preferred acidic alpha-amylase is derived from a strain Aspergillus niger. In a preferred embodiment the acid fungal alpha-amylase is the one from A. niger disclosed as “AMYA_ASPNG” in the Swiss-prot/TeEMBL database under the primary accession no. P56271 and described in WO 89/01969 (Example 3).

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

In a preferred embodiment, the alpha-amylase is derived from Aspergillus kawachii and disclosed by Kaneko et al., 1996, J. Ferment. Bioeng. 81: 292-298, “Molecular-cloning and determination of the nucleotide-sequence of a gene encoding an acid-stable alpha-amylase from Aspergillus kawachii”; and further as EMBL:#AB008370.

The fungal alpha-amylase may also be a wild-type enzyme comprising a starch-binding domain (SBD) and an alpha-amylase catalytic domain (i.e., non-hybrid), or a variant thereof. In an embodiment the wild-type alpha-amylase is derived from a strain of Aspergillus kawachii.

In a further embodiment, the alpha amylase is a fungal acid alpha-amylase is a hybrid alpha-amylase. Preferred examples of fungal hybrid alpha-amylases include the ones disclosed in WO 2005/003311 or U.S. application publication no. 2005/0054071 (Novozymes) or U.S. application No. 60/638,614 (Novozymes) which is hereby incorporated by reference. A hybrid alpha-amylase may comprise an alpha-amylase catalytic domain (CD) and a carbohydratebinding domain/module (CBM), such as a starch binding domain, and optional a linker.

Specific examples of contemplated hybrid alpha-amylases include those disclosed in Table 1 to 5 of the examples in U.S. application No. 60/638,614, including Fungamyl variant with catalytic domain JA118 and Athelia rolfsii SBD (SEQ ID NO:100 in U.S. 60/638,614), Rhizomucor pusillus alpha-amylase with Athelia rolfsii AMG linker and SBD (SEQ ID NO:101 in U.S. 60/638,614), Rhizomucor pusillus alpha-amylase with Aspergillus niger glucoamylase linker and SBD (which is disclosed in Table 5 as a combination of amino acid sequences SEQ ID NO: 20, SEQ ID NO: 72 and SEQ ID NO: 96 in U.S. application Ser. No. 11/316,535) or as V039 in Table 5 in WO 2006/069290, and Meripilus giganteus alpha-amylase with Athelia rolfsii glucoamylase linker and SBD (SEQ ID NO: 102 in U.S. application No. 60/638,614). Other specifically contemplated hybrid alpha-amylases are any of the ones listed in Tables 3, 4, 5, and 6 in Example 4 in U.S. application Ser. No. 11/316,535 and WO 2006/069290 (hereby incorporated by reference).

Other specific examples of contemplated hybrid alpha-amylases include those disclosed in U.S. application publication no. 2005/0054071, including those disclosed in Table 3 on page 15, such as Aspergillus niger alpha-amylase with Aspergillus kawachii linker and starch binding domain.

Bacterial alpha-amylases useful in the processes according to the invention include alpha-amylases derived from a strain of the genus Bacillus, such as Bacillus licheniformis, Bacillus stearothermophilus.

Contemplated are also alpha-amylases which exhibit a high identity to any of the above mentioned alpha-amylases, i.e., more than 70%, more than 75%, more than 80%, more than 85% more than 90%, more than 95%, more than 96%, more than 97%, more than 98%, more than 99% or even 100% identity to the mature enzyme sequences.

Preferred commercial compositions comprising alpha-amylase include MYCOLASE™ (DSM), BAN™, TERMAMYL™ SC, FUNGAMYL™, LIQUOZYME™ X, LIQUOZYME™ SC and SAN™ SUPER, SAN™ EXTRA L (Novozymes NS) and CLARASE™ L-40,000, DEX-LO™, SPEZYME™ FRED, SPEZYME™ AA, SPEZYME™ ALPHA, SPEZYME™ DELTA AA, GC358, GC980, SPEZYME™ CL and SPEZYME™ RSL (DuPont-Genencor), FUELZYME™ (from Verenium Corp, USA).

These alpha amylases may, if desired, also be included in the compositions according to the invention at the very low amounts set forth above.

Glucoamylases and pullulanases which are useful in the processes according to the present invention include those disclosed above in relation to the compositions according to the invention.

Hence, in particular embodiments of the invention the alpha amylase is selected from Aspergillus niger, Aspergillus terreus or Rhizomucor pusillus alpha amylases, the glucoamylase is selected from Aspergillus niger, Aspergillus fumigatus, Talaromyces emersonii, Trametes cingulata, Trichoderma reesei, Humicloa insolens and Gloeophyllum trabeum glucoamylases, and the pullulanase is selected from Bacillus deramificans or Bacillus acidopullulyticus pullulanases.

Specifically, the glucoamylase may comprise an amino acid sequence selected from the group consisting of
i) The amino acid sequence set forth in any of SEQ ID NOs: 2-12 and 27 or a mature polypeptide thereof;
ii) A subsequence of the amino acid sequence set forth in any one of SEQ ID NOs: 2-12 and 27 or of said mature polypeptide thereof;
iii) A variant amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii).

In related embodiments, the glucoamylase consists essentially of an amino acid sequence as set forth in any of items i)-iii) above. In other related embodiments the glucoamylase consists of an amino acid sequence as set forth in any of items i)-iii) above.

The alpha-amylase used in the process according to the invention comprises/consists essentially of/consists of an amino acid sequence selected from the group consisting of
i) The amino acid sequence set forth in any one of SEQ ID NOs: 1 and 17-26 or a mature polypeptide thereof;
ii) A subsequence of the amino acid sequence set forth in any one of SEQ ID NOs: 1 and 17-26 or of said mature polypeptide thereof;
iii) A variant amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii).

In related embodiments, the alpha-amylase consists essentially of an amino acid sequence as set forth in any of items i)-iii) above. In other related embodiments the alpha-amylase consists of an amino acid sequence as set forth in any of items i)-iii) above.

In particular embodiments, the alpha-amylase comprising or consisting of the amino acid sequence defined in iii) is a variant of an alpha-amylase comprising or consisting of the amino sequence defined in SEQ ID NO: 20 or a mature polypeptide thereof, wherein the following mutations have been made: 1181*/G182*/N193F (using the amino acid numbering in SEQ ID NO: 20).

According to other embodiments, the alpha-amylase comprising or consisting of the amino acid sequence defined in iii) is a variant of an alpha-amylase comprising or consisting of the amino sequence defined in SEQ ID NO: 23 or a mature polypeptide thereof, wherein the following mutations have been made: H156Y+A181T+N190F+A209V+Q264S (using the amino acid numbering in SEQ ID NO: 21).

In even further embodiments, the alpha-amylase comprising or consisting of the amino acid sequence defined in iii) is a variant of an alpha-amylase comprising or consisting of the amino sequence defined in SEQ ID NO: 23 or a mature polypeptide thereof, wherein the following mutations have been made: G48A+T49I+G107A+H156Y+A181T+N190F+1201F+A209V+Q264S (using the numbering in SEQ ID NO: 21).

The pullulanase used in the process according to the invention may comprises/consists essentially of/consists of an amino acid sequence selected from the group consisting of
i) The amino acid sequence set forth in any of SEQ ID NOs: 13-16 or a mature polypeptide thereof;
ii) A subsequence of the amino acid sequence set forth in any one of SEQ ID NOs: 13-16 or of said mature polypeptide thereof;
iii) A variant amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii).

In related embodiments, the pullulanase consists essentially of an amino acid sequence as set forth in any of items i)-iii) above. In other related embodiments the pullulanase consists of an amino acid sequence as set forth in any of items i)-iii) above.

Within the scope of the present invention are embodiments wherein the glucoamylase comprises or consists of an amino acid sequence selected from the group consisting of:
i) the amino acid sequence set forth in SEQ ID NO: 12 or a mature polypeptide thereof,
ii) a subsequence of the amino acid sequence set forth in SEQ ID NO: 12 or of said mature polypeptide thereof, and
iii) a variant amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii);

wherein the pullulanase comprises or consists of an amino acid sequence selected from the group consisting of:
iv) the amino acid sequence set forth in any one of SEQ ID NOs: 13-16 or a mature polypeptide thereof;
v) a subsequence of the amino acid sequence set forth in any one of SEQ ID NOs: 13-16 or of said mature polypeptide thereof, and
vi) a variant amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in iv) and v); and

wherein the alpha-amylase comprises or consists of an amino acid sequence selected from the group consisting of:
vii) the amino acid sequence set forth in SEQ ID NO: 18 or a mature polypeptide thereof,
viii) a subsequence of the amino acid sequence set forth in SEQ ID NO: 18 or of said mature polypeptide thereof, and
iv) a variant amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in vii) and viii).

When said glucoamylase is a subsequence or a variant amino acid sequence as defined above, it preferably has at least 80%, such as at least 90% of the glucoamylase activity of the respective amino acid of which it is a subsequence or variant, when tested as set forth above in relation to the definition of “Glucoamylase activity (AGU)”.

When said pullulanase is a subsequence or a variant amino acid sequence as defined above, it preferably has at least 80%, such as at least 90% of the pullulanase activity of the respective amino acid of which it is a subsequence or variant, when tested as set forth above in relation to the definition of “Pullulanase activity (NPUN)”.

When the alpha-amylase defined above is a subsequence or a variant, it preferably has at least 80%, such as at least 90% of the alpha-amylase activity of the respective alpha-amylase selected from SEQ ID NOs: 1 and 17-26 or of the mature polypeptide thereof, of which it is a subsequence or variant, when tested as set forth above in relation to the definition of “Acid alpha-Amylase Units (FAU(A))” or “Alpha-amylase Activity (KNU)”: In the present context an alpha-amylase comprising an amino acid sequences selected from the group consisting of SEQ ID NOs: 1, 17-19 and 26 or of a mature polypeptide thereof is considered to be a fungal alpha amylase and activity is tested as provided in relation to the above definition of “Acid alphaAmylase Units (FAU(A))”. An alpha-amylase comprising of an amino acid sequences selected from the group consisting of SEQ ID NOs: 20-25 or of a mature polypeptide thereof, is considered to be a bacterial alpha amylase and activity is tested as provided in relation to the above definition of “Alpha-amylase Activity (KNU)”.

In a further aspect, the invention provides a process for producing glucose syrup from liquefied starch. The process comprises contacting the liquefied starch with a composition according to according to the invention a described hereinabove.

The liquefied starch may in particular contain from 25-45% dry solids (% DS) (w/w %), such as from 25-40% DS (w/w %), from 30-38% DS, from 32-38% DS, or such as from 34-36% DS.

In further embodiments, the process comprises subjecting the liquified starch to incubation under conditions allowing starch hydrolysis/saccharification.

### Items

1. A composition comprising a glucoamylase and a pullulanase, wherein the ratio of pullulanase activity expressed as NPUN/g to glucoamylase activity expressed as AGU/g is higher than 6, such as higher than 7, higher than 8, higher than 9 or within the range of 6-20, such as within the range of 6-19, within the range of 6-18. within the range of 6-17, within the range of 6-16, within the range of 6-15, within the range of 6-14, within the range of 6-13, within the range of 6-12, within the range of 6-11, within the range of 7-20, within the range of 7-19, within the range of 7-18, within the range of 7-17, within the range of 7-16, within the range of 7-15, within the range of 7-14, within the range of 7-13, within the range of 7-12, within the range of 7-11, within the range of 8-20, within the range of 8-19, within the range of 8-18, within the range of 8-17, within the range of 8-16, within the range of 8-15, within the range of 8-14, within the range of 8-13, within the range of 8-12, within the range of 8-11, within the range of 9-20, within the range of 9-19, within the range of 9-18, within the range of 9-17, within the range of 9-16, within the range of 9-15, within the range of 9-14, within the range of 9-13, within the range of 9-12 or such as within the range of 9-11.
2. The composition according to item 1, wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 29, such as higher than 50, higher than 100, higher than 200, higher than 300, higher than 400, higher than 500, higher than 600, higher than 800, higher than 1000, higher than 2000, higher than 3000, higher than 4000 or such as higher than 5000, and wherein if the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 29, such as higher than 50, higher than 100, higher than 200, higher than 300, higher than 400, higher than 500, higher than 600, higher than 800, higher than 1000, higher than 2000, higher than 3000, higher than 4000 or such as higher than 5000.
3. The composition according to item 1 or 2, wherein the ratio of pullulanase activity expressed as NPUN/g to glucoamylase activity expressed as AGU/g is higher than 6, and wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 100, and wherein if the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 100.
4. The composition according to item 1 or 2, wherein the ratio of pullulanase activity expressed as NPUN/g to glucoamylase activity expressed as AGU/g is within the range of 6-20, and wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 100, and wherein if the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 100.
5. The composition according to item 1 or 2, wherein the ratio of pullulanase activity expressed as NPUN/g to glucoamylase activity expressed as AGU/g is within the range of 6-20, and wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 500, and wherein if the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 500
6. The composition according to item 1 or 2, wherein the ratio of pullulanase activity expressed as NPUN/g to glucoamylase activity expressed as AGU/g is within the range of 7-15, and wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 100, and wherein if the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 100.
7. The composition according to item 1 or 2, wherein the ratio of pullulanase activity expressed as NPUN/g to glucoamylase activity expressed as AGU/g is within the range of 7-15, and wherein if said composition comprises a polypeptide having alpha-amylase activity and that polypeptide is of fungal origin, then the ratio of glucoamylase activity expressed as AGU/g to alpha-amylase activity expressed as Fungal alpha-Amylase Units FAU(A)/g is higher than 500, and wherein if the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 100.
8. The composition according to any of the preceding items, wherein said glucoamylase is selected from the group consisting of an Aspergillus niger glucoamylase, a Talaromyces emersonii glucoamylase, a Trametes cingulata glucoamylase a Gloeophyllum trabeum glucoamylase, a Trichoderma Reesei glucoamylase, a Humicola insolens glucoamylase and an Aspergillus fumigatus glucoamylase, and hybrids and variants thereof.
9. The composition according to any of the preceding items, wherein said pullulanase is selected from the group consisting of a Bacillus deramificans pullulanase, a Bacillus acidopullulyticus pullulanase, and hybrids and variants thereof.
10. The composition according to any of the preceding items, wherein the glucoamylase comprises/consists essentially of/consists of an amino acid sequence selected from the group consisting of
   i) The amino acid sequence set forth in any of SEQ ID NO: 2-12 and 27;
   ii) A subsequence of the amino acid sequence set forth in any one of SEQ ID NOs: 2-12 and 27;
   iii) An amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii).
11. The composition according to any of the preceding items, wherein the alpha-amylase comprises/consists essentially of/consists of an amino acid sequence selected from the group consisting of
   i) The amino acid sequence set forth in any of SEQ ID NOs: 1 and 17-26;
   ii) A subsequence of the amino acid sequence set forth in any one of SEQ ID NOs: 1 and 17-26;
   iii) An amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii).
12. The composition according to any of the preceding items, wherein the pullulanase comprises/consists essentially of/consists of an amino acid sequence selected from the group consisting of
   i) The amino acid sequence set forth in any one of SEQ ID NOs: 13-16;
   ii) A subsequence of the amino acid sequence set forth in any one of SEQ ID NOs: 13-16;
   iii) An amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii).
13. The composition according to any of the preceding items, said composition being a liquid or solid composition.
14. A process for producing glucose syrup comprising,
   i) providing a composition comprising liquefied starch, and adding to said composition a glucoamylase (AMG) and a pullulanase;
   ii) subjecting the composition to incubation under conditions allowing starch hydrolysis/saccharification, and subsequently
   iii) adding an alpha-amylase to the said composition and subjecting the composition to incubation under conditions allowing starch hydrolysis/saccharification.
15. The process according to item 14, wherein the amounts of glucoamylase added in step i) of item 14 correspond to 0.05-0.5 AGU/gDS, such as 0.05-0.4 AGU/gDS, 0.05-0.35 AGU/gDS, 0.05-0.3 AGU/gDS, 0.075-0.5 AGU/gDS, 0.075-0.4 AGU/gDS, 0.075-0.35 AGU/gDS 0.075-0.3 AGU/gDS, 0.075-0.5 AGU/gDS 0.1-0.5 AGU/gDS, 0.1-0.4 AGU/gDS, 0.1-0.35 AGU/gDS, 0.1-0.3 AGU/gDS, 0.125-0.5 AGU/gDS, 0.125-0.4 AGU/gDS, 0.125-0.35 AGU/gDS, 0.125-0.3 AGU/gDS, 0.15-0.5 AGU/gDS, 0.15-0.4 AGU/gDS, 0.15-0.35 AGU/gDS, 0.15-0.3 AGU/gDS, 0.175-0.5 AGU/gDS, 0.175-0.4 AGU/gDS, 0.175-0.35 AGU/gDS, 0.175-0.3 AGU/gDS, 0.2-0.5 AGU/gDS, 0.2-0.4 AGU/gDS, 0.2-0.35 AGU/gDS, 0.2-0.3 AGU/gDS, 0.24-0.5 AGU/gDS, 0.24-0.4 AGU/gDS, 0.24-0.35 AGU/gDS or such as 0.24-0.26 AGU/gDS.
16. The process according to any of items 14-15, wherein the amounts of pullulanase added in step i) of item 14 correspond to 0.05-5 NPUN(X)/gDS, such as 0.05-4.5 NPUN(X)/gDS, 0.05-4 NPUN(X)/gDS, 0.05-3.5 NPUN(X)/gDS, 0.05-3 NPUN(X)/gDS, 0.05-2.5 NPUN(X)/gDS, 0.1-5 NPUN(X)/gDS, 0.1-4 NPUN(X)/gDS, 0.1-3.5 NPUN(X)/gDS, 0.1-3 NPUN(X)/gDS, 0.1-2.5 NPUN(X)/gDS, 0.25-5 NPUN(X)/gDS, 0.25-4.5 NPUN(X)/gDS, 0.25-4 NPUN(X)/gDS, 0.25-3.5 NPUN(X)/gDS, 0.25-3 NPUN(X)/gDS, 0.5-5 NPUN(X)/gDS, 0.5-4.5 NPUN(X)/gDS, 0.5-4 NPUN(X)/gDS, 0.5-3.5 NPUN(X)/gDS, 0.05-3 NPUN(X)/gDS, 0.5-2.5 NPUN(X)/gDS, 1.0-5 NPUN(X)/gDS, 1.0-4.5 NPUN(X)/gDS, 1.0-4 NPUN(X)/gDS, 1.0-3.5 NPUN(X)/gDS, 1.0-3 NPUN(X)/gDS, 1.0-2.5 NPUN(X)/gDS, 1.5-5 NPUN(X)/gDS, 1.5-4.5 NPUN(X)/gDS, 1.5-4 NPUN(X)/gDS, 1.5-3.5 NPUN(X)/gDS, 1.5-3 NPUN(X)/gDS, 1.5-2.5 NPUN(X)/gDS, 2-5 NPUN(X)/gDS, 2-4.5 NPUN(X)/gDS, 2-4 NPUN(X)/gDS, 2-3.5 NPUN(X)/gDS, 2-3 NPUN(X)/gDS, 2-5 NPUN(X)/gDS, 2-2.5 NPUN(X)/gDS, 2.25-5 NPUN(X)/gDS, 2.25-4.5 NPUN(X)/gDS, 2.25-4 NPUN(X)/gDS, 2.25-3.5 NPUN(X)/gDS, 2.25-3 NPUN(X)/gDS, 2.25-2.5 NPUN(X)/gDS, or such as 2.50-2.54 NPUN(X)/gDS.
17. The process according to any of items 14-16, wherein the amounts of alpha-amylase added in step iii) of item 14 correspond to 0.0005-0.025 (FAU)(A)/gDS, such as 0.0005-0.0225 (FAU)(A)/gDS, 0.0005-0.02 (FAU)(A)/gDS, 0.0005-0.0175 (FAU)(A)/gDS, 0.0005-0.015 (FAU)(A)/gDS, 0.0005-0.0125 (FAU)(A)/gDS, 0.001-0.025 (FAU)(A)/gDS, 0.001-0.0225 (FAU)(A)/gDS, 0.001-0.02 (FAU)(A)/gDS, 0.001-0.0175 (FAU)(A)/gDS, 0.001-0.015 (FAU)(A)/gDS, 0.001-0.0125 (FAU)(A)/gDS, 0.0025-0.025 (FAU)(A)/gDS, 0.0025-0.0225 (FAU)(A)/gDS, 0.0025-0.02 (FAU)(A)/gDS, 0.0025-0.0175 (FAU)(A)/gDS, 0.0025-0.015 (FAU)(A)/gDS, 0.0025-0.0125 (FAU)(A)/gDS, 0.005-0.025 (FAU)(A)/gDS, 0.005-0.0225 (FAU)(A)/gDS, 0.005-0.02 (FAU)(A)/gDS, 0.005-0.0175 (FAU)(A)/gDS, 0.005-0.015 (FAU)(A)/gDS, 0.005-0.0125 (FAU)(A)/gDS, 0.0075-0.025 (FAU)(A)/gDS, 0.0075-0.0225 (FAU)(A)/gDS, 0.0075-0.02 FAU(A)/gDS, 0.0075-0.0175 FAU(A)/gDS, 0.0075-0.015 FAU(A)/gDS, 0.0075-0.0125 FAU(A)/gDS, 0.008-0.025 (FAU)(A)/gDS, 0.008-0.0225 (FAU)(A)/gDS, 0.008-0.02 (FAU)(A)/gDS, 0.008-0.0175 (FAU)(A)/gDS, 0.008-0.015 (FAU)(A)/gDS, 0.008-0.0125 (FAU)(A)/gDS, 0.009-0.0125 (FAU)(A)/gDS or such as 0.01 FAU(A)/gDS, and the amounts of alpha-amylase added in step iii) of item 14 correspond to 0.0005-0.025 KNU/gDS, such as 0.0005-0.0225 KNU/gDS, 0.0005-0.02 KNU/gDS, 0.0005-0.0175 KNU/gDS, 0.0005-0.015 KNU/gDS, 0.0005-0.0125 KNU/gDS, 0.001-0.025 KNU/gDS, 0.001-0.0225 KNU/gDS, 0.001-0.02 KNU/gDS, 0.001-0.0175 KNU/gDS, 0.001-0.015 KNU/gDS, 0.001-0.0125 KNU/gDS, 0.0025-0.025 KNU/gDS, 0.0025-0.0225 KNU/gDS, 0.0025-0.02 KNU/gDS, 0.0025-0.0175 KNU/gDS, 0.0025-0.015 KNU/gDS, 0.0025-0.0125 KNU/gDS, 0.005-0.025 KNU/gDS, 0.005-0.0225 KNU/gDS, 0.005-0.02 KNU/gDS, 0.005-0.0175 KNU/gDS, 0.005-0.015 KNU/gDS, 0.005-0.0125 KNU/gDS, 0.0075-0.025 KNU/gDS, 0.0075-0.0225 KNU/gDS, 0.0075-0.02 KNU/gDS, 0.0075-0.0175 KNU/gDS, 0.0075-0.015 KNU/gDS, 0.0075-0.0125 KNU/gDS, 0.008-0.025 KNU/gDS, 0.008-0.0225 KNU/gDS, 0.008-0.02 KNU/gDS, 0.008-0.0175 KNU/gDS, 0.008-0.015 KNU/gDS, 0.008-0.0125 KNU/gDS, 0.009-0.01 KNU/gDS or such as 0.0095 KNU/gDS. if said alpha-amylase is a bacterial alpha-amylase.
18. The process according to any of items 14-17, wherein the amounts of glucoamylase added in step i) of item 14 correspond to 0.05-0.5 AGU/gDS, the amounts of pullulanase added in step i) of item 14 correspond to 0.05-5 NPUN(X)/gDS, and the amounts of alpha-amylase added in step iii) of item 14 correspond to 0.0005-0.025 (FAU)(A)/gDS if the alpha-amylase is a fungal alpha-amylase and to 0.0005-0.025 KNU/gDS if said alpha-amylase is a bacterial alpha-amylase.
19. The process according to any of items 14-17, wherein the amounts of glucoamylase added in step i) of item 14 correspond to 0.125-0.35 AGU/gDS, the amounts of pullulanase added in step i) of item 14 correspond to 1.5-3.5 NPUN(X)/gDS, and the amounts of alpha-amylase added in step iii) of item 14 correspond to 0.0025-0.02 (FAU)(A)/gDS if the alpha-amylase is a fungal alpha-amylase and to 0.0025-0.02 KNU/gDS if said alpha-amylase is a bacterial alpha-amylase.
20. The process according to any of items 14-17, wherein the amounts of glucoamylase added in step i) of item 14 correspond to 0.175-0.3 AGU/gDS, the amounts of pullulanase added in step i) of item 14 correspond to 2-4 NPUN(X)/gDS, and the amounts of alpha-amylase added in step iii) of item 14 correspond to 0.0075-0.0175 FAU(A)/gDS if the alpha-amylase is a fungal alpha-amylase and to 0.0075-0.0175 KNU/gDS if said alpha-amylase is a bacterial alpha-amylase.
21. The process according to any of items 14-17, wherein the amounts of glucoamylase added in step i) of item 14 correspond to 0.2-0.3 AGU/gDS, the amounts of pullulanase added in step i) of item 14 correspond to 2-3 NPUN(X)/gDS, and the amounts of alpha-amylase added in step iii) of item 14 correspond to 0.008-0.0125 (FAU)(A)/gDS if the alpha-amylase is a fungal alpha-amylase and to 0.008-0.0125 KNU/gDS if said alpha-amylase is a bacterial alpha-amylase.
22. The process according to any of items 14-21, comprising adding to said composition comprising liquefied starch a formulation comprising a glucoamylase and a pullulanase, wherein if said formulation comprises a polypeptide having alpha-amylase activity, wherein if said composition comprises a polypeptide having alpha-amylase activity and the polypeptide is of fungal origin, then the ratio of glucoamylase activity expressed as AGU/g to alpha-amylase activity expressed as Fungal alpha-Amylase Units FAU(A)/g in said composition is higher than 29, such as higher than 50, higher than 100, higher than 200, higher than 300, higher than 400, higher than 500, higher than 600, higher than 800, higher than 1000, higher than 2000, higher than 3000, higher than 4000, or such as higher than 5000, and wherein if the polypeptide having alpha-amylase activity is of bacterial origin, then the ratio of glucoamylase activity expressed as AGU/g to alpha-amylase activity expressed as Kilo Novo alpha-amylase Units (KNU)/g is higher than 29, such as higher than 50, higher than 100, higher than 200, higher than 300, higher than 400, higher than 500, higher than 600, higher than 800, higher than 1000, higher than 2000, higher than 3000, higher than 4000, or such as higher than 5000.
23. The process according to any of items 14-21, wherein the glucoamylase and the pullulanase added in step ii) are in a composition as defined in any of items 1-13.
24. The process according to any of items 14-23, wherein said incubation in step ii) of item 14 has a duration of from 2-35 hours, such as from 2-30 hours, from 2-28 hours, from 2-26 hours, from 2-24 hours, from 2-22 hours, from 2-20 hours, from 2-18 hours, from 2-16 hours, from 2-14 hours, from 2-12 hours, from 2-10 hours, from 2-8 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-35 hours, from 10-30 hours, from 10-28 hours, from 10-26 hours, from 10-24 hours, from 10-22 hours, from 10-20 hours, from 10-18 hours, from 10-16 hours, from 10-14 hours, from 10-12 hours, from 14-35 hours, from 14-30 hours, from 14-28 hours, from 14-26 hours, from 14-24, from 14-22 hours, from 14-20 hours, from 14-18 hours, from 14-16 hours, from 18-35 hours, from 18-30 hours, from 18-28 hours, from 18-26 hours, from 18-24, from 18-22 hours, from 18-20 hours, from 20-35 hours, from 20-30 hours, from 20-28 hours, from 20-26 hours, from 20-24, from 20-22 hours, form 22-35 hours, from 22-30 hours, from 22-28 hours, from 22-26 hours, from 22-24 hours or such or sus as a duration of 24 hours.
25. The process according to any of items 14-24, wherein said incubation in step iii) of item 14 has a duration of from 2-94 hours, such as from 2-90 hours, from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-94 hours, from 4-90 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-94 hours, from 10-90 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-94 hours, from 14-90 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-94 hours, from 20-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-94 hours, from 24-90 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-94 hours, from 30-90 hours, from 30-80 hours, from 30-70 hours, from 30-60, from 30-55 hours, from 30-50 hours, from 30-45 hours, from 30-40 hours, from 30-35 hours, from 40-94 hours, from 40-90 hours, from 40-80 hours, from 40-70 hours, from 40-60, from 40-55 hours, from 40-50 hours, from 40-45 hours, from 50-94 hours, from 50-90 hours, from 50-80 hours, from 50-70 hours, from 50-60, from 60-94 hours, from 60-90 hours, from 60-80 hours, or such as a duration of from 60-70 hours.
26. The process according to any of items 14-25, wherein said incubation in step ii) of item 14 has a duration of from 2-35 hours and said incubation in step iii) of item 14 has a duration of from 2-94 hours, such as from 2-90 hours, from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-94 hours, from 4-90 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-94 hours, from 10-90 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-94 hours, from 14-90 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-94 hours, from 20-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-94 hours, from 24-90 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-94 hours, from 30-90 hours, from 30-80 hours, from 30-70 hours, from 30-60, from 30-55 hours, from 30-50 hours, from 30-45 hours, from 30-40 hours, from 30-35 hours, from 40-94 hours, from 40-90 hours, from 40-80 hours, from 40-70 hours, from 40-60, from 40-55 hours, from 40-50 hours, from 40-45 hours, from 50-94 hours, from 50-90 hours, from 50-80 hours, from 50-70 hours, from 50-60, from 60-94 hours, from 60-90 hours, from 60-80 hours, or such as a duration of from 60-70 hours.
27. The process according to any of items 14-25, wherein said incubation in step ii) of item 14 has a duration of from 2-20 hours and said incubation in step iii) of item 14 has a duration of from 2-94 hours, such as from 2-90 hours, from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-94 hours, from 4-90 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-94 hours, from 10-90 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-94 hours, from 14-90 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-94 hours, from 20-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-94 hours, from 24-90 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-94 hours, from 30-90 hours, from 30-80 hours, from 30-70 hours, from 30-60, from 30-55 hours, from 30-50 hours, from 30-45 hours, from 30-40 hours, from 30-35 hours, from 40-94 hours, from 40-90 hours, from 40-80 hours, from 40-70 hours, from 40-60, from 40-55 hours, from 40-50 hours, from 40-45 hours, from 50-94 hours, from 50-90 hours, from 50-80 hours, from 50-70 hours, from 50-60, from 60-94 hours, from 60-90 hours, from 60-80 hours, or such as a duration of from 60-70 hours.
28. The process according to any of items 14-25, wherein said incubation in step ii) of item 14 has a duration of from 4-35 hours and said incubation in step iii) of item 14 has a duration of from 2-92 hours, such as from 2-90 hours, from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-92 hours, from 4-90 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-92 hours, from 10-90 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-92 hours, from 14-90 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-92 hours, from 20-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-92 hours, from 24-90 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-92 hours, from 30-90 hours, from 30-80 hours, from 30-70 hours, from 30-60, from 30-55 hours, from 30-50 hours, from 30-45 hours, from 30-40 hours, from 30-35 hours, from 40-92 hours, from 40-90 hours, from 40-80 hours, from 40-70 hours, from 40-60, from 40-55 hours, from 40-50 hours, from 40-45 hours, from 50-92 hours, from 50-90 hours, from 50-80 hours, from 50-70 hours, from 50-60, from 60-94 hours, from 60-90 hours, from 60-80 hours, or such as a duration of from 60-70 hours.
29. The process according to any of items 14-25, wherein said incubation in step ii) of item 14 has a duration of from 4-22 hours, and said incubation in step iii) of item 14 has a duration of from 2-92 hours, such as from 2-90 hours, from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-92 hours, from 4-90 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-92 hours, from 10-90 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-92 hours, from 14-90 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-92 hours, from 20-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-92 hours, from 24-90 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-92 hours, from 30-90 hours, from 30-80 hours, from 30-70 hours, from 30-60, from 30-55 hours, from 30-50 hours, from 30-45 hours, from 30-40 hours, from 30-35 hours, from 40-92 hours, from 40-90 hours, from 40-80 hours, from 40-70 hours, from 40-60, from 40-55 hours, from 40-50 hours, from 40-45 hours, from 50-92 hours, from 50-90 hours, from 50-80 hours, from 50-70 hours, from 50-60, from 60-94 hours, from 60-90 hours, from 60-80 hours, or such as a duration of from 60-70 hours.
30. The process according to any of items 14-25, wherein said incubation in step ii) of item 14 has a duration of from 10-35 hours, and said incubation in step iii) of item 14 has a duration of from 2-86 hours, such as from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-86 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-86 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-86 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-86 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-86 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-86 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-86 hours, from 30-80 hours, from 30-70 hours, from 30-60, from 30-55 hours, from 30-50 hours, from 30-45 hours, from 30-40 hours, from 30-35 hours, from 40-86 hours, from 40-80 hours, from 40-70 hours, from 40-60, from 40-55 hours, from 40-50 hours, from 40-45 hours, from 50-86 hours, from 50-80 hours, from 50-70 hours, from 50-60, from 60-94 hours, from 60-90 hours, from 60-80 hours, or such as a duration of from 60-70 hours.
31. The process according to any of items 14-25, wherein said incubation in step ii) of item 14 has a duration of from 10-24 hours, and said incubation in step iii) of item 14 has a duration of from 2-86 hours, such as from 2-80 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-86 hours, from 4-80 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-86 hours, from 10-80 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-86 hours, from 14-80 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-86 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-86 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-86 hours, from 24-80 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-86 hours, from 30-80 hours, from 30-70 hours, from 30-60, from 30-55 hours, from 30-50 hours, from 30-45 hours, from 30-40 hours, from 30-35 hours, from 40-86 hours, from 40-80 hours, from 40-70 hours, from 40-60, from 40-55 hours, from 40-50 hours, from 40-45 hours, from 50-86 hours, from 50-80 hours, from 50-70 hours, from 50-60, from 60-94 hours, from 60-90 hours, from 60-80 hours, or such as a duration of from 60-70 hours.
32. The process according to any of items 14-25, wherein said incubation in step ii) of item 14 has a duration of from 14-35 hours, and said incubation in step iii) of item 14 has a duration of from 2-70 hours, such as from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-94 hours, from 25-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-70 hours, from 30-60, from 30-55 hours, from 30-50 hours, from 30-45 hours, from 30-40 hours, from 30-35 hours, from 40-70 hours, from 40-60, from 40-55 hours, from 40-50 hours, from 40-45 hours, from 50-70 hours, from 50-60, from 60-94 hours, from 60-90 hours, from 60-80 hours, or such as a duration of from 60-70 hours.
33. The process according to any of items 14-25, wherein said incubation in step ii) of item 14 has a duration of from 14-26 hours, and said incubation in step iii) of item 14 has a duration of from 2-70 hours, such as from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-94 hours, from 25-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24 hours, from 20-22 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-70 hours, from 30-60, from 30-55 hours, from 30-50 hours, from 30-45 hours, from 30-40 hours, from 30-35 hours, from 40-70 hours, from 40-60, from 40-55 hours, from 40-50 hours, from 40-45 hours, from 50-70 hours, from 50-60, from 60-94 hours, from 60-90 hours, from 60-80 hours, or such as a duration of from 60-70 hours.
34. The process according to any of items 14-25, wherein said incubation in step ii) of item 14 has a duration of from 18-35 hours, and said incubation in step iii) of item 14 has a duration of from 2-78 hours, such as from 2-74 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-78 hours, from 4-74 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-76 hours, from 10-74 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-78 hours, from 14-74 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-78 hours, from 20-74 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-78 hours, from 24-76 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-78 hours, from 30-74 hours, from 30-70 hours, from 30-60, from 30-55 hours, from 30-50 hours, from 30-45 hours, from 30-40 hours, from 30-35 hours, from 40-76 hours, from 40-74 hours, from 40-70 hours, from 40-60, from 40-55 hours, from 40-50 hours, from 40-45 hours, from 50-78 hours, from 50-74 hours, from 50-70 hours, from 50-60, from 60-94 hours, from 60-90 hours, from 60-80 hours, or such as a duration of from 60-70 hours.
35. The process according to any of items 14-25, wherein said incubation in step ii) of item 14 has a duration of from 18-28 hours, and said incubation in step iii) of item 14 has a duration of from 2-78 hours, such as from 2-74 hours, from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-78 hours, from 4-74 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-76 hours, from 10-74 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24, from 10-22 hours, from 10-20 hours, from 10-18 hours, from 10-16 hours, from 10-14 hours, from 10-12 hours, from 14-78 hours, from 14-74 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-78 hours, from 20-74 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-78 hours, from 24-76 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-78 hours, from 30-74 hours, from 30-70 hours, from 30-60, from 30-55 hours, from 30-50 hours, from 30-45 hours, from 30-40 hours, from 30-35 hours, from 40-76 hours, from 40-74 hours, from 40-70 hours, from 40-60, from 40-55 hours, from 40-50 hours, from 40-45 hours, from 50-78 hours, from 50-74 hours, from 50-70 hours, from 50-60, from 60-94 hours, from 60-90 hours, from 60-80 hours, or such as a duration of from 60-70 hours.
36. The process according to any of items 14-25, wherein said incubation in step ii) of item 14 has a duration of from 20-35 hours, and said incubation in step iii) of item 14 has a duration of from 2-76 hours, such as from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-76 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24 hours, from 4-22 hours, from 4-20 hours, from 4-18 hours, from 4-16 hours, from 4-14 hours, from 4-12 hours, from 4-10 hours, from 4-8 hours, from 10-76 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24 ours, from 10-22 hours, from 10-20 hours, from 10-18 hours, from 10-16 hours, from 10-14 hours, from 10-12 hours, from 14-76 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-76 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-76 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-76 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-76 hours, from 30-70 hours, from 30-60, from 30-55 hours, from 30-50 hours, from 30-45 hours, from 30-40 hours, from 30-35 hours, from 40-76 hours, from 40-70 hours, from 40-60, from 40-55 hours, from 40-50 hours, from 40-45 hours, from 50-76 hours, from 50-70 hours, from 50-60, from 60-94 hours, from 60-90 hours, from 60-80 hours, or such as a duration of from 60-70 hours.
37. The process according to any of items 14-22, wherein said incubation in step ii) of item 14 has a duration of from 20-30 hours, and said incubation in step iii) of item 14 has a duration of from 2-76 hours, such as from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-76 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-76 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24 hours, from 10-22 hours, from 10-20 hours, from 10-18 hours, from 10-16 hours, from 10-14 hours, from 10-12 hours, from 14-76 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-76 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-76 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-76 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-76 hours, from 30-70 hours, from 30-60, from 30-55 hours, from 30-50 hours, from 30-45 hours, from 30-40 hours, from 30-35 hours, from 40-76 hours, from 40-70 hours, from 40-60, from 40-55 hours, from 40-50 hours, from 40-45 hours, from 50-76 hours, from 50-70 hours, from 50-60, from 60-94 hours, from 60-90 hours, from 60-80 hours, or such as a duration of from 60-70 hours.
38. The process according to any of items 14-25, wherein said incubation in step ii) of item 14 has a duration of from 22-35 hours, and said incubation in step iii) of item 14 has a duration of from 2-74 hours, such as from 2-70 hours, from 2-60, from 2-55 hours, from 2-50 hours, from 2-45 hours, from 2-40 hours, from 2-35 hours, from 2-30 hours, from 2-28 hours, from 2-26 hours, from 2-24, from 2-22 hours, from 2-20 hours, from 2-18 hours, from 2-16 hours, from 2-14 hours, from 2-12 hours, from 2-10 hours, from 2-8 hours, from 4-74 hours, from 4-70 hours, from 4-60, from 4-55 hours, from 4-50 hours, from 4-45 hours, from 4-40 hours, from 4-35 hours, from 4-30 hours, from 4-28 hours, from 4-26 hours, from 4-24, from 4-22 hours, from 4-20 hours, from 4-18 hours, from 4-16 hours, from 4-14 hours, from 4-12 hours, from 4-10 hours, from 4-8 hours, from 10-74 hours, from 10-70 hours, from 10-60, from 10-55 hours, from 10-50 hours, from 10-45 hours, from 10-40 hours, from 10-35 hours, from 10-30 hours, from 10-28 hours, from 10-26 hours, from 10-24 hours, from 10-22 hours, from 10-20 hours, from 10-18 hours, from 10-16 hours, from 10-14 hours, from 10-12 hours, from 14-74 hours, from 14-70 hours, from 14-60, from 14-55 hours, from 14-50 hours, from 14-45 hours, from 14-40 hours, from 14-35 hours, from 14-30 hours, from 14-28 hours, from 14-26 hours, from 14-24, from 14-22 hours, from 14-20 hours, from 14-18 hours, from 14-16 hours, from 20-74 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 25-94 hours, from 25-90 hours, from 20-80 hours, from 20-70 hours, from 20-60, from 20-55 hours, from 20-50 hours, from 20-45 hours, from 20-40 hours, from 20-35 hours, from 20-30 hours, from 20-28 hours, from 20-26 hours, from 20-24, from 20-22 hours, from 24-74 hours, from 24-70 hours, from 24-60, from 24-55 hours, from 24-50 hours, from 24-45 hours, from 24-40 hours, from 24-35 hours, from 24-30 hours, from 24-28 hours, from 24-26 hours, from 30-94 hours, from 30-90 hours, from 30-80 hours, from 30-70 hours, from 30-60, from 30-55 hours, from 30-50 hours, from 30-45 hours, from 30-40 hours, from 30-35 hours, from 40-74 hours, from 40-70 hours, from 40-60, from 40-55 hours, from 40-50 hours, from 40-45 hours, from 50-94 hours, from 50-90 hours, from 50-80 hours, from 50-70 hours, from 50-60, from 60-74 hours, or such as a duration of from 60-70 hours.
39. The process according to any of items 14-38, wherein the incubation in step iii) of item 14 is allowed to proceed until the amount of glucose (% DX) in said composition has reached a level corresponding to 95% (w/w) or more, such as 95.5% (w/w) or more, 95.75% (w/w) or more, 96% (w/w) or more, 96.25% (w/w) or more, or such as 96.75% (w/w) or more.
40. The process according to any of items 14-39, wherein the duration of the process from addition of said a glucoamylase (AMG) and said pullulanase to the composition comprising liquefied starch until termination of the starch hydrolysis/saccharification is from 24-96 hours, such as from 24-72 hours, from 24-64 hours, from 24-48 hours, from 24-36 hours, from 24-32 hours, from 32-96 hours, from 32-72 hours, from 32-64 hours, from 32-48 hours, from 32-36 hours, from 36-96 hours, from 36-72 hours, from 36-64 hours, from 36-48 hours, from 48-96 hours, such as from 48-72 hours, from 48-64 hours, from 56-96 hours, such as from 56-72 hours, or such as from 56-64 hours,
41. The process according to any of items 14-40, wherein the starch hydrolysis/saccharification takes place at a pH which is within the range of 3.5-5.0, such as at pH in the range of 4.0-4.5, and at a temperature, which is within the range of 59−70° C., such as in the range of 59−65° C. or such as in the range of 59−62° C.
42. The process according to any of items 14-41, wherein the composition comprising liquefied starch provided in step i) of item 14 contains from 25-45% dry solids (% DS), such as from 25-40% DS, from 28-38% DS, from 30-38% DS, or such as from 33-36% DS.
43. The process according to any of items 14-42, wherein the alpha amylase is selected from Bacillus licheniformis, Bacillus stearothermophilus, Aspergillus niger, Aspergillus terreus or Rhizomucor pusillus alpha amylases, the glucoamylase is selected from Aspergillus niger, Aspergillus fumigatus, Talaromyces emersonii, Trametes cingulata, Trichoderma reesei, Humicloa insolens and Gloeophyllum trabeum glucoamylases, and the pullulanase is selected from Bacillus deramificans or Bacillus acidopullulyticus pullulanases.
44. The process according to any of items 14-43, wherein the glucoamylase comprises/consists essentially of/consists of an amino acid sequence selected from the group consisting of
   iv) The amino acid sequence set forth in any of SEQ ID NO: 2-12 and 27;
   v) A subsequence of the amino acid sequence set forth in any one of SEQ ID NOs: 2-12 and 27;
   vi) An amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii).
45. The process according to any of items 14-44, wherein the alpha-amylase comprises/consists essentially of/consists of an amino acid sequence selected from the group consisting of
   iv) The amino acid sequence set forth in any of SEQ ID NOs: 1 and 17-26;
   v) A subsequence of the amino acid sequence set forth in any one of SEQ ID NOs: 1 and 17-26;
   vi) An amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii).
46. The process according to any of items 14-45, wherein the pullulanase comprises/consists essentially of/consists of an amino acid sequence selected from the group consisting of
   iv) The amino acid sequence set forth in any one of SEQ ID NOs: 13-16;
   v) A subsequence of the amino acid sequence set forth in any one of SEQ ID NOs: 13-16;
   vi) An amino acid sequence, which has at least 70%, such as at least 75%, at least 80%, at least 85%, at least 90%, at least 91%, at least 92%, at least 93%, at least 94%, at least 95%, at least 96%, at least 97%, at least 98%, at least 99%, such as at least 99.5% sequence identity to any one of the amino acids sequences set forth in i) and ii).
47. A process for producing glucose syrup from liquefied starch, comprising contacting the liquefied starch with a composition according to any of items 1-13.
48. The process according to item 47, wherein said liquefied starch contains from 25-45% dry solids (% DS), such as from 25-40% DS, from 30-38% DS, from 32-38% DS, or such as from 34-36% DS.
49. The process according to any of items 47-48, said process comprising subjecting the liquified starch to incubation under conditions allowing starch hydrolysis/saccharification.

The present invention is further described by the following examples that should not be construed as limiting the scope of the invention.

### EXAMPLES

### Example 1

Maltodextrin powder from corn starch liquefaction was dissolved in water while heating to make slurry at 34.2% dry solids. The solid content of the slurry was measured using Refractive index measurement showing 1.39221. The slurry was adjusted to a pH of 4.3 using a 1M Hydrochloric acid solution. 18 gram aliquots of this slurry were added to 18 glass reaction scintillation vials with septum cap closures and were inserted in a heating block to be heated to a temperature of 61° C. Each vial was given an enzyme dosage based on the table below and additional water was added to each vial to reach a target dry solid of 33%. 1.5 mL samples were taken via needles through the septum from each vial at different time points (36 hour and 48 hours) and were deactivated at 105° C. for 5 minutes. 1 mL of each deactivated sample was diluted with 4 mL deionized water. The diluted samples were evaluated using a HPLC method DP1-4 for measuring dextrose purity (% DP1 or % DX, % DP2, % DP3 and % DP4+).

Results from Table 1 show the higher the NPUN/AGU ratio, the higher the percent dextrose.

Table 1 shows that constant AGU dose of 0.25 AGU/gDS, when the ratio of NPUN to AGU (NPUN/AGU) increased in the blend from 10.1 to 30.2, an increase in % DX at peak was observed (96.7 to 97.0% DX). The newly created blends with NPUN/AGU of 9 or more show 0.7 to 1% DX improvement over commercial products. When the NPUN/g DS dose in the blend was kept as constant, by varying the AGU/gDS dose, higher % DX was not observed, only the speed of reaction was affected. The benefit in % DX in blends with NPUN/AGU>9 is not only due to use of a lower isomaltose forming AMG (JGA98) showing lower DP2 of 2.1-2.2% at 36 hours but also is due to lower % DP3 and DP4+ at 36 hours.

TABLE 1
Dextrose content

| | | | NPUN/ | | | | | |
|---|---|---|---|---|---|---|---|---|
| | AGU/ | NPUN/ | AGU | % DX | % DX | % DP2 | % DP3 | % DP4+ |
| Enzyme | gDS | gDS | ratio | at 36 hr | at 48 hr | at 36 hr | at 36 hr | at 36 hr |
| Dextrozyme DX 2X | 0.18 | 0.36 | 2 | 96.0 | 96.0 | 2.4 | 0.8 | 0.8 |
| Dextrozyme DX Plus | 0.18 | 0.72 | 4 | 96.1 | 95.9 | 2.4 | 0.8 | 0.7 |
| AMG (SEQ ID NO: 1) + | 0.28 | 2.52 | 9 | 96.6 | 96.7 | 2.2 | 0.5 | 0.7 |
| Pullulanase (SEQ ID NO: 13 | | | | | | | | |
| (0.28/2.52) | | | | | | | | |
| AMG (SEQ ID NO: 1) + | 0.25 | 2.52 | 10.1 | 96.5 | 96.7 | 2.2 | 0.5 | 0.8 |
| Pullulanase (SEQ ID NO: 13 | | | | | | | | |
| (0.25/2.52) | | | | | | | | |
| AMG (SEQ ID NO: 1) + | 0.23 | 2.52 | 11.0 | 96.5 | 96.7 | 2.1 | 0.5 | 0.9 |
| Pullulanase (SEQ ID NO: 13 | | | | | | | | |
| (0.23/2.52) | | | | | | | | |
| AMG (SEQ ID NO: 1) + | 0.25 | 3.6 | 14.4 | 96.5 | 96.7 | 2.2 | 0.5 | 0.7 |
| Pullulanase (SEQ ID NO: 13 | | | | | | | | |
| (0.25/3.6) | | | | | | | | |
| AMG (SEQ ID NO: 1) + | 0.25 | 5.04 | 20.2 | 96.9 | 96.9 | 2.2 | 0.5 | 0.5 |
| Pullulanase (SEQ ID NO: 13 | | | | | | | | |
| (0.25/5.04) | | | | | | | | |
| AMG (SEQ ID NO: 1) + | 0.25 | 7.56 | 30.2 | 97.0 | 96.7 | 2.1 | 0.5 | 0.4 |
| Pullulanase (SEQ ID NO: 13 | | | | | | | | |
| (0.25/7.56) | | | | | | | | |

### Example 2

Maltodextrin powder from corn starch liquefaction was dissolved in water while heating to make slurry starch slurry at 37.8% dry solids. The solid content of the slurry was measured using Refractive index measurement showing 1.39964. The slurry was adjusted to a pH of 4.3 using a 1M Hydrochloric acid solution. 18 gram aliquots of this slurry were added to 18 glass reaction scintillation vials with septum cap closures and were inserted in a heating block to be heated to a temperature of 61° C. Each vial was given an enzyme dosage based on the table below and additional water was added to each vial to reach a target dry solid of 30, 33 or 36%. 1.5 mL samples were taken via needles through the septum from each vial at different time points and were deactivated at 105° C. for 5 minutes. 1 mL of each deactivated sample was diluted with 4 mL deionized water. The diluted samples were evaluated using a HPLC method DP1-4 for measuring dextrose purity (% DP1 or % DX).

Table 2 shows the dextrose purity at 48 hours at the three different solids. The new enzyme blend shows 0.8-0.9% DX improvement over commercial enzyme blend at all solids tested. This results show that with new enzyme blend at the same solids higher DX (0.8-0.9% DX improvement) can be achieved. Also similar dextrose purity can be achieved at higher solids. For example using the new enzyme blend a % DX of 96.5% DX can be achieved at 36% DS instead of 30% DS using Dextrozyme DX 2×.

TABLE 2
Dextrose purity

| | Dose | | | % DX at 48 hours (Peak time) | | |
|---|---|---|---|---|---|---|
| Enzyme | AGU/gDS | NPUN/gDS | NPUN/AGU | 30% DS | 33% DS | 36% DS |
| Dextrozyme DX | 0.18 | 0.36 | 2 | 96.5 | 96.1 | 95.6 |
| 2X | | | | | | |
| AMG (SEQ ID | 0.25 | 2.52 | 10.08 | 97.4 | 96.9 | 96.5 |
| NO: 12) + | | | | | | |
| Pullulanase | | | | | | |
| (SEQ ID NO: 13) | | | | | | |

### Example 3

Saccharification by enzyme blends with delayed dosing of a fungal alpha-amylase, (SEQ ID NO: 15), was evaluated to see how delayed dosing of alpha-amylase affects the DX and the speed of saccharification.

### Materials and Methods

Saccharification test was carried out using a Gilson Liquid handler, with which 36% DS of DE11 maltodextrin made via Liquozyme Supra from was pH adjusted to 4.3 and was saccharified at 60° C. Prior to saccharification any residual alpha-amylase activity in the maltodextrin was inactivated by heat treatment. At different time points, the ratio of DX (glucose), DP2, DP3 and DP4+ of the syrup was analyzed by HPLC.

The initial enzyme dosages are shown in Table 1. “Benchmark” is a conventional enzyme blend for saccharification, containing glucoamylase, pullulanase and apha-amylase, the latter being a side activity of the glucoamylase. “AMG+Pul” is a blend of glucoamylase with no alpha-amylase side activity and pullulanase. The sample volume increased by the alpha-amylase spike was only 0.2% so that the change in DS is negligible.

After 24 h incubation with AMG+Pul blend, purified alpha-amylase (SEQ ID NO: 15) was dosed to be 0.01 FAU(A)/gDS, and compared with the benchmark enzymes. The dosages and the timings of enzymes are shown in Table 1.

TABLE 3
Enzyme dosages

| | | | | | Alpha- | Alpha- | |
|---|---|---|---|---|---|---|---|
| | | | | Pullulanase | amylase | amylase | |
| | | | | SEQ ID | SEQ ID NO: 1 | SEQ ID NO: | |
| | | | | NO: 13 | (side activity) | 15 spiked | Spiked |
| | Enzyme | Glucoamylase | [AGU/ | [NPUN | [FAU(A)/ | [FAU(A)/ | Spiked |
| Sample | blend | (AMG) | gDS] | (XP)/gDS] | gDS] | gDS] | at |
| 1 | Bench- | SEQ ID NO: 1 | 0.18 | 0.36 | 0.045 | — | — |
| | mark | | | | | | |
| 2 | AMG + Pul | SEQ ID NO: 12 | 0.194 | 2.52 | — | — | — |
| | blend | | | | | | |
| 3 | AMG + Pul | SEQ ID NO: 12 | 0.194 | 2.52 | — | 0.01 | 24 h |
| | blend, AA | | | | | | |
| | spiked | | | | | | |

### Results

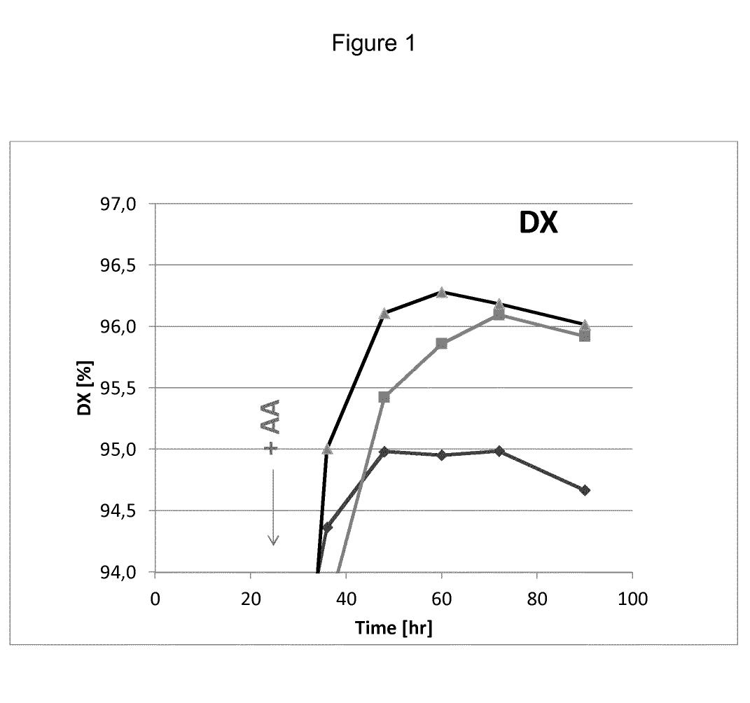

The DX curves from two experiments (FIG. 1) and the summary table (Table 2) show that the spike of alpha-amylase at 24 h both improved the saccharification speed and the peak DX significantly, compared to AMG+Pul blend. When compared at 48 h (saccharification period employed by many plants), the improvement of DX by the spike was as high as 0.4%-0.6%.

TABLE 4
Peak DX, Peak time and DX@48 h of AMG + Pul
blend spiked with AA, compared with benchmarks

| | Peak DX | Peak Time | DX@48 h |
|---|---|---|---|
| | [%] | [hr] | [%] |
| Benchmark | 95.0 | 48 | 95.0 |
| AMG + Pul blend | 96.1 | 72 | 95.7 |
| AMG + Pul blend, AA spiked | 96.3 | 60 | 96.1 |

### CONCLUSION

The results presented above confirm that the delayed dosing of Alpha-amylase to a blend of glucoamylase and pullulanase can further improve both DX and saccharification speed.

### Example 4

Maltodextrin powder from corn starch liquefaction was dissolved in water while heating to make slurry starch slurry at 37.2% dry solids. The solid content of the slurry was measured using Refractive index measurement showing 1.3982. The slurry was adjusted to a pH of 4.3 using a 1M Hydrochloric acid solution. 18 gram aliquots of this slurry were added to 18 glass reaction scintillation vials with septum cap closures and were inserted in a heating block to be heated to a temperature of 61° C. Each vial was given an enzyme dosage based on the table below and additional water was added to each vial to reach a target dry solid of 36%. 1.5 mL samples were taken via needles through the septum from each vial at different time points and were deactivated at 105° C. for 5 minutes. 1 mL of each deactivated sample was diluted with 4 mL deionized water. The diluted samples were evaluated using a HPLC method DP1-4 for measuring dextrose purity (% DP1 or % DX).

Table 5 shows the dextrose purity of syrup at different hours. The results show that addition of bacterial alpha amylase when NPUN/KNUT is more than 60, has a positive effect on speed and % DX.

TABLE 5
Dextrose purity

| | AGU/ | NPUN/ | AFAU/ | KNU/ | NPUN/ | NPUN/ | NPUN/ | % DX at different hour | | | | |
|---|---|---|---|---|---|---|---|---|---|---|---|---|
| Enter sample times | gDS | gDS | gDS | gDS | AGU | AFAU | KNU | 24 hr | 36 hr | 42 hr | 48 hr | 60 hr |
| Dextrozyme DX 2X | 0.18 | 0.36 | 0 | 0 | 2 | N.A. | N.A. | 93.3 | 95.3 | 95.4 | 95.4 | 95.4 |
| Dextrozyme DX 2X + | 0.18 | 0.36 | 0 | 0.075 | 2 | N.A. | 4.8 | 94.1 | 95.4 | 95.5 | 95.4 | 95.4 |
| Alpha amylase | | | | | | | | | | | | |
| (SEQ ID NO: 25) | | | | | | | | | | | | |
| AMG (SEQ ID NO: 1) + | 0.25 | 0.96 | 0.001 | 0 | 3.84 | 960 | N.A. | 93.3 | 95.6 | 95.8 | 95.8 | 95.8 |
| Pullulanase | | | | | | | | | | | | |
| (SEQ ID NO: 13) | | | | | | | | | | | | |
| AMG (SEQ ID NO: 1) + | 0.25 | 0.96 | 0.001 | 0.0375 | 3.84 | 960 | 25.6 | 93.5 | 95.7 | 95.8 | 95.7 | 95.7 |
| Pullulanase | | | | | | | | | | | | |
| (SEQ ID NO: 13) + | | | | | | | | | | | | |
| Alpha amylase | | | | | | | | | | | | |
| (SEQ ID NO: 25) | | | | | | | | | | | | |
| AMG (SEQ ID NO: 1) + | 0.25 | 0.96 | 0.001 | 0.0750 | 3.84 | 960 | 12.8 | 93.3 | 95.6 | 95.6 | 95.7 | 95.8 |
| Pullulanase | | | | | | | | | | | | |
| (SEQ ID NO: 13) + | | | | | | | | | | | | |
| Alpha amylase | | | | | | | | | | | | |
| (SEQ ID NO: 25) | | | | | | | | | | | | |
| AMG (SEQ ID NO: 12) + | 0.25 | 2.52 | 0 | 0 | 10.08 | N.A. | N.A. | 93.1 | 95.9 | 96.1 | 96.1 | 96.2 |
| Pullulanase | | | | | | | | | | | | |
| (SEQ ID NO: 13) | | | | | | | | | | | | |
| AMG (SEQ ID NO: 12) + | 0.25 | 2.52 | 0 | 0.0375 | 10.08 | N.A. | 67.2 | 94.6 | 96.0 | 96.1 | 96.2 | 96.1 |
| Pullulanase | | | | | | | | | | | | |
| (SEQ ID NO: 13) + | | | | | | | | | | | | |
| Alpha amylase | | | | | | | | | | | | |
| (SEQ ID NO: 25) | | | | | | | | | | | | |
| AMG (SEQ ID NO: 12) + | 0.25 | 2.52 | 0 | 0.0750 | 10.08 | N.A. | 33.6 | 94.5 | 96.0 | 96.0 | 96.1 | 96.1 |
| Pullulanase | | | | | | | | | | | | |
| (SEQ ID NO: 13) + | | | | | | | | | | | | |
| Alpha amylase | | | | | | | | | | | | |
| (SEQ ID NO: 25) | | | | | | | | | | | | |

### Example 5

Saccharification by enzyme blends with delayed dosing of fungal alpha-amylases was evaluated to see how the timing of alpha-amylase addition affects the DX during saccharification.

Saccharification test was carried out using a Gilson Liquid handler, with which 36% DS of DE11 maltodextrin made via Liquozyme Supra was pH adjusted to 4.3 and was saccharified at 60° C. Prior to saccharification any residual alpha-amylase activity in the maltodextrin was inactivated by heat treatment.

Initially, a blend of glucoamylase (SEQ ID NO: 12) and pullulanase (SEQ ID NO: 13) containing no alpha-amylase side activity (AMG+Pul blend) was added to be 0.194 AGU/gDS for glucoamylase and 2.25 NPUN/gDS for pullulanase. At the same time, the DS of the maltodextrin syrup became 33% by dilution caused by the enzyme addition. After different periods (0-24 h) of incubation, purified alpha-amylase (SEQ ID NO: 18) was dosed to be 15 μg/gDS corresponding to 0.01 FAU(A)/gDS, and compared with the control without alpha-amylase dosing

(“No AA addition”). The sample volume increase by the alpha-amylase spike was only 0.2% so that the change in DS is negligible. After 36 h from the start of incubation with AMG+Pul blend, 0.75 ml aliquot of the sample was taken and the ratio of DX (glucose), DP2, DP3 and DP4+ of the syrup was analyzed by HPLC. The timings of alpha-amylase dosing and the DX at 36 h are shown in Table 6.

TABLE 6
Timings of alpha-amylase (SEQ ID NO: 18)
dosing and the resultant DX at 36 h

| AA dosed at | DX@36 h |
|---|---|
| No AA addition | 94.9 |
| 0 h | 95.4 |
| 2 h | 95.6 |
| 4 h | 95.9 |
| 8 h | 96.0 |
| 16 h | 96.1 |
| 24 h | 96.0 |

In the same experiment batch, another purified fungal alpha-amylase AM4279 purified from Aspergillus terreus (mature polypeptide of SEQ ID NO: 26) was spiked to be 15 μg/gDS instead of the alpha amylase set forth in SEQ ID NO: 18 at 0 h and 24 h. The timings of alpha-amylase dosing and the DX at 36 h are shown in Table 7.

TABLE 7
Timings of alpha-amylase (mature polypeptide of SEQ
ID NO: 26) dosing and the re-sultant DX at 36 h.

| AA dosed at | DX@36 h |
|---|---|
| No AA addition | 94.9 |
| 0 h | 95.2 |
| 24 h | 95.4 |

In the same experiment batch, another AMG+Pul blend with glucoamylase from Aspergillus niger (mature polypeptide of SEQ ID NO: 27) instead of the glucoamylase set forth in SEQ ID NO: 12 was initially dosed to be 0.18 AGU/gDS for glucoamylase while the pullulanase dosage was kept to be 2.25 NPUN/gDS. The timings of alpha-amylase (SEQ ID NO: 18) dosing and the DX at 36 h are shown in Table 8.

TABLE 8
Timings of alpha-amylase (SEQ ID NO: 18)
dosing and the resultant DX at 36 h

| AA dosed at | DX@36 h |
|---|---|
| No AA addition | 94.9 |
| 0 h | 95.9 |
| 24 h | 96.2 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A composition comprising a glucoamylase and a pullulanase, wherein the ratio of pullulanase activity expressed as NPUN/g to glucoamylase activity expressed as AGU/g is higher than 6.

2.-9. (canceled)

10. The composition according to claim 1, wherein the glucoamylase comprises/consists essentially of/consists of an amino acid sequence selected from the group consisting of
i) the amino acid sequence set forth in any one of SEQ ID NO: 2-12 and 27 or a mature polypeptide thereof;
ii) a subsequence of the amino acid sequence set forth in any one of SEQ ID NOs: 2-12 and 27 or of said mature polypeptide thereof;
iii) an amino acid sequence, which has at least 70% sequence identity to any one of the amino acids sequences set forth in i) and ii).

11. The composition according to claim 1, wherein the alpha-amylase comprises/consists essentially of/consists of an amino acid sequence selected from the group consisting of
i) the amino acid sequence set forth in any one of SEQ ID NOs: 1 and 17-26 or a mature polypeptide thereof;
ii) a subsequence of the amino acid sequence set forth in any one of SEQ ID NOs: 1 and 17-26 or of said mature polypeptide thereof;
iii) an amino acid sequence, which has at least 70% sequence identity to any one of the amino acids sequences set forth in i) and ii).

12. The composition according to claim 1, wherein the pullulanase comprises/consists essentially of/consists of an amino acid sequence selected from the group consisting of
i) the amino acid sequence set forth in any one of SEQ ID NOs: 13-16 or a mature polypeptide thereof;
ii) a subsequence of the amino acid sequence set forth in any one of SEQ ID NOs: 13-16 or of said mature polypeptide thereof;
iii) an amino acid sequence, which has at least 70% sequence identity to any one of the amino acids sequences set forth in i) and ii).

13. The composition according to claim 1, said composition being a liquid or solid composition.

14. A process for producing glucose syrup comprising,
i) providing a composition comprising liquefied starch, and adding to said composition a glucoamylase (AMG) and a pullulanase;
ii) subjecting the composition to incubation under conditions allowing starch hydrolysis/saccharification, and subsequently
iii) adding an alpha-amylase to the said composition and subjecting the composition to incubation under conditions allowing starch hydrolysis/saccharification.

15. The process according to claim 14, wherein the amount of glucoamylase added in step i) corresponds to 0.05-0.5 AGU/gDS.

16. The process according to claim 14, wherein the amount of pullulanase added in step i) corresponds to 0.05-5 NPUN(X)/gDS.

17. The process according to claim 14, wherein the amount of alpha-amylase added in step iii) corresponds to 0.0005-0.025 (FAU)(A)/gDS if said alpha-amylase is a fungal alpha-amylase and to 0.0005-0.025 KNU/gDS if said alpha-amylase is a bacterial alpha-amylase.

18.-23. (canceled)

24. The process according to claim 14, wherein said incubation in step ii) of claim 14 has a duration of from 2-35 hours.

25. The process according to claim 14, wherein said incubation in step iii) of claim 14 has a duration of from 2-94 hours.

26.-38. (canceled)

39. The process according to claim 14, wherein the incubation in step iii) is allowed to proceed until the amount of glucose (% DX) in said composition has reached a level corresponding to 95% (w/w) or more.

40. The process according to claim 14, wherein the duration of the process from addition of said a glucoamylase (AMG) and said pullulanase to the composition comprising liquefied starch until termination of the starch hydrolysis/saccharification is from 24-96 hours.

41. The process according to claim 14, wherein the starch hydrolysis/saccharification takes place at a pH which is within the range of 3.5-5.0, and at a temperature, which is within the range of 59-70° C.

42. The process according to claim 14, wherein the composition comprising liquefied starch provided in step i) contains from 25-45% dry solids (% DS).

43. (canceled)

44. The process according to claim 14, wherein the glucoamylase comprises/consists essentially of/consists of an amino acid sequence selected from the group consisting of
i) the amino acid sequence set forth in any of SEQ ID NO: 2-12 and 27 or a mature polypeptide thereof;
ii) a subsequence of the amino acid sequence set forth in any one of SEQ ID NOs: 2-12 and 27 or of said mature polypeptide thereof;
iii) an amino acid sequence, which has at least 70% sequence identity to any one of the amino acids sequences set forth in i) and ii).

45. The process according to claim 14, wherein the alpha-amylase comprises/consists essentially of/consists of an amino acid sequence selected from the group consisting of
i) the amino acid sequence set forth in any of SEQ ID NOs: 1 and 17-26 or a mature polypeptide thereof;
ii) a subsequence of the amino acid sequence set forth in any one of SEQ ID NOs: 1 and 17-26 or of said mature polypeptide thereof;
iii) an amino acid sequence, which has at least 70% sequence identity to any one of the amino acids sequences set forth in i) and ii).

46. The process according to claim 14, wherein the pullulanase comprises/consists essentially of/consists of an amino acid sequence selected from the group consisting of
i) the amino acid sequence set forth in any one of SEQ ID NOs: 13-16 or a mature polypeptide thereof;
ii) a subsequence of the amino acid sequence set forth in any one of SEQ ID NOs: 13-16 or of said mature polypeptide thereof;
iii) an amino acid sequence, which has at least 70% sequence identity to any one of the amino acids sequences set forth in i) and ii).

47. A process for producing glucose syrup from liquefied starch, comprising contacting the liquefied starch with a composition according to claim 1.

48. The process according to claim 47, wherein said liquefied starch contains from 25-45% dry solids (% DS), such as from 25-40% DS from 30-38% DS, from 32-38% DS, or such as from 34-36% DS.
